(19) United States
(12) Patent Application Publication
(10) Pub. No.: US 2014/0039029 A1
(43) Pub. Date: Feb. 6, 2014

(54) PREPARATION AND USE OF (+)-1-(3,4-DICHLOROPHENYL)-3-AZABICYCLO[3.1.0]HEXANE IN THE TREATMENT OF CONDITIONS AFFECTED BY MONOAMINE NEUROTRANSMITTERS

(71) Applicants: Anthony Alexander McKinney, San Diego, CA (US); Frank Bymaster, Brownsburg, IN (US); Walter Piskorski, Nashua, NH (US)

(72) Inventors: Anthony Alexander McKinney, San Diego, CA (US); Frank Bymaster, Brownsburg, IN (US); Walter Piskorski, Nashua, NH (US)

(21) Appl. No.: 13/923,016

(22) Filed: Jun. 20, 2013

Related U.S. Application Data
(63) Continuation of application No. 13/310,694, filed on Dec. 2, 2011, now abandoned.
(60) Provisional application No. 61/419,769, filed on Dec. 3, 2010.

(51) Int. Cl. A61K 31/403; A61K 45/06

(52) U.S. Cl. CPC A61K 31/403; A61K 45/06
USPC 514/412

(57) ABSTRACT

The present invention relates to (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane and their use alone or in combination with additional psychotherapeutic compositions in the treatment of conditions affected by monoamine neurotransmitters, including treatment of refractory individuals.

### RELATED APPLICATIONS

This application claims priority benefit of U.S. Provisional patent application Ser. No. 61/419,769, filed Dec. 3, 2010, the disclosure of which is incorporated herein in its entirety by reference.

### TECHNICAL FIELD

The present invention relates to selective inhibition of the reuptake of monoamine neurotransmitters. Specifically, the present invention relates to compositions comprising (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane and their use in the treatment of conditions affected by monoamine neurotransmitters.

### BACKGROUND OF THE INVENTION

Drug development has generally focused on affecting a specific target molecule in order to minimize side effects and increase potency. However, clinical studies of disorders ranging from cancer to schizophrenia have indicated that drugs affecting a variety of targets may be more efficacious (Frantz et al., 2005). In the treatment of depression, the use of serotonin-norepinephrine reuptake inhibitors have been shown to lead to higher remission rates than the use of selective serotonin reuptake inhibitors alone (Thase et al., 2001) and combinations of selective serotonin reuptake inhibitors with dopamine and norepinephrine inhibitors can be more effective than administration of a selective serotonin reuptake inhibitor by itself (Trivedi et al., 2006).

Triple reuptake inhibitors selectively inhibit the reuptake of multiple monoamine neurotransmitters. Specifically, they inhibit the reuptake of 5-hydroxytryptamine (serotonin), norepinephrine and dopamine by blocking the action of the serotonin transporter, norepinephrine transporter and dopamine transporter. There are several triple reuptake inhibitors under investigation for use in the treatment of a variety of conditions including depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain and Alzheimer's disease. (See, e.g. Mcmillen et al., 2007; Gardner et al, 2006; Tizzano et al. 2008; Basile et al., 2007).

1-(3,4-dichlorophenyl)-3-azabicyclo[3.1.0]hexane is a triple reuptake inhibitor currently under investigation. It exhibits chirality and has two enantiomers. Enantiomers may have the same or different effects on biological entities and many pharmaceutical agents are sold as racemates even though the desired or any pharmacological activity resides in only one enantiomer. For example, the S(+)-methacholine enantiomer is 250 times more potent than the R(−) enantiomer. With ketamine, the (S)-enantiomer is an anesthetic, but the (R)-enantiomer is a hallucinogen. Administration of a racemic mixture of any drug can be disadvantageous in that racemic mixtures may be less pharmacologically active than one of the enantiomers as in the case of methacholine, or it may have increased toxicity or other undesirable side effects as in ketamine.

According to the World Health Organization, depression is the leading cause of disability and the fourth leading contributor to the global burden of disease (World Health Organization). It affects more than 121 million people worldwide. Two-thirds of patients who are initially prescribed antidepressant medications do not experience a timely remission (Fava et al., 1996). For those who fail to respond to initial treatment there is no clear treatment protocol. Residual symptoms are associated with an increased risk of relapse, impaired social and occupational functioning, and chronicity of course (Judd et al., 1998). There is therefore an unmet need for the identification of effective pharmaceuticals which may be used in the treatment of depression and other conditions affected by monoamine neurotransmitters, particularly for individuals that were unresponsive to initial therapies.

### SUMMARY OF EXEMPLARY EMBODIMENTS

Provided herein are compositions and methods using an unbalanced triple reuptake inhibitor, (+)-1-(3,4-dichlorophenyl)-3-azabicyclo[3.1.0]hexane as shown below, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane, for the treatment of mammals, including humans, suffering from signs and symptoms of disorders generally treated with triple reuptake inhibitors including, but not limited to, depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain states, and Alzheimer's disease. Unbalanced as used herein refers to the relative effects on each of the monoamine transporters. In this case reference is made to a triple reuptake inhibitor with the most activity against the serotonin transporter, half as much to the norepinephrine transporter and one eighth to the dopamine transporter. In contrast, a balanced triple reuptake inhibitor would have similar activity against each of the three monoamine transporters.

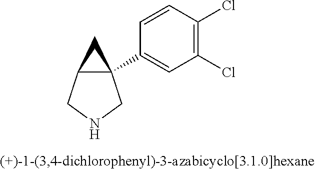 [SMILES: Clc1ccc([C@]23CNC[C@H]2C3)cc1Cl]

(+)-1-(3,4-dichlorophenyl)-3-azabicyclo[3.1.0]hexane agents as used herein are substantially free of the corresponding (−) enantiomer, (−)-1-(3,4-dichlorophenyl)-3-azabicyclo[3.1.0]hexane. In addition to being enantiomeric, (+)-1-(3,4-dichlorophenyl)-3-azabicyclo[3.1.0]hexane exists in at least three polymorphic forms, labeled herein polymorphs A, B and C. The polymorphs may be used in pharmaceutical compositions in combination or in forms that are substantially free of one or more of the other polymorphic forms.

(+)-1-(3,4-dichlorophenyl)-3-azabicyclo[3.1.0]hexane may furthermore be in the form of pharmaceutically acceptable active salts, glycosylated derivatives, metabolites, solvates, hydrates and/or prodrugs. For example, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. Additionally, pharmaceutically acceptable forms may include inorganic and organic acid addition salts such as hydrochloride salt.

Additional background information regarding (±)-1-(3,4-dichlorophenyl)-3-azabicyclo[3.1.0]hexane, such as binding studies, may be found, for example, in U.S. Pat. No. 4,435,419, WO/20040466457, WO2007127396, WO02066427, WO2006023659, U.S. patent application Ser. No. 11/740,667, and U.S. Pat. No. 6,372,919, each of which is incorporated herein by reference in their entirety.

Additionally provided herein are combinatorial compositions and coordinate treatment means using additional or secondary psychotherapeutic agents in combination with (+)-1-(3,4-dichlorophenyl)-3-azabicyclo[3.1.0]hexane agents including (+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane. Suitable secondary psychotherapeutic drugs for use in the compositions and methods herein include, but are not limited to, drugs from the general classes of anti-convulsant, mood-stabilizing, anti-psychotic, anxiolytic, benzodiazepines, calcium channel blockers, anti-inflammatories, and antidepressants. (See, e.g., R J. Baldessarini in Goodman & Gilman's The Pharmacological Basis of Therapeutics, 11th Edition, Chapters 17 and 18, McGraw-Hill, 2005 for a review). Exemplary antidepressants include, for example, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, norepinephrine-dopamine reuptake inhibitors, serotonin-norepinephrine reuptake inhibitors, multiple monoamine reuptake inhibitors, monoamine oxidase inhibitors, atypical antidepressants, atypical antipsychotics, anticonvulsants, or opiate agonists.

It is shown herein that use of (+)-1-(3,4-dichlorophenyl)-3-azabicyclo[3.1.0]hexane agents ((+)-1-(3,4-dichlorophenyl)-3-azabicyclo[3.1.0]hexane, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating, preventing, alleviating, or moderating disorders affected by monoamine neurotransmitters or biogenic amines, specifically disorders that are alleviated by inhibiting dopamine and/or norepinephrine and/or serotonin reuptake. Such conditions include, but are not limited to, depression, anxiety, panic disorder, post-traumatic stress disorder, obsessive compulsive disorder, schizophrenia and allied disorders, obesity, tic disorders, ADHD, substance abuse disorders, Parkinson's disease, chronic pain states, and Alzheimer's disease. Use of the compositions of the present invention may increase monoamine neurotransmitter levels and/or selectively inhibit reuptake of monoamine neurotransmitters and/or biogenic amines.

The unbalanced serotonin-norepinephrine-dopamine reuptake inhibition ratio of ˜1:2:8, respectively, of (+)-1-(3,4-dichlorophenyl)-3-azabicyclo[3.1.0]hexane. (Skolnick et al., 2003) allows for higher dosages of (+)-1-(3,4-dichlorophenyl)-3-azabicyclo[3.1.0]hexane to be used without triggering the dopaminergic or norepinephrine side effects such as elevated heart rate, increased blood pressure, gastrointestinal (nausea/vomiting and constipation/diarrhea) effects, dry mouth, insomnia, anxiety, and hypomania seen in similar dosages of balanced triple reuptake inhibitors or unbalanced triple reuptake inhibitors with different inhibition ratios.

The compositions herein are also unexpectedly useful in the treatment of individuals who have previously been treated one or more times for disorders affected by monoamine neurotransmitters, particularly depression. (+)-1-(3,4-dichlorophenyl)-3-azabicyclo[3.1.0]hexane agents have shown unexpected efficacy in the treatment of individuals who have been refractory to previous treatments for disorders affected by monoamine neurotransmitters, i.e. individuals that have not responded or have responded in an unsatisfactory manner to at least one other treatment, specifically anti-depressants such as, but not limited to, tri-cyclic antidepressants (TCAs), specific monoamine reuptake inhibitors, selective serotonin reuptake inhibitors including citalopram, selective norepinephrine or noradrenaline reuptake inhibitors, serotonin-norepinephrine reuptake inhibitors, selective dopamine reuptake inhibitors, norepinephrine-dopamine reuptake inhibitors, multiple monoamine reuptake inhibitors, monoamine oxidase inhibitors, atypical antidepressants, atypical antipsychotics, anticonvulsants, ant-inflammatories or opiate agonists. Individuals may have been refractory to previous treatment(s) for any reason. In some embodiments, refractory individuals may have failed to respond or failed to respond sufficiently to a previous treatment. In one embodiment, a refractory individual may have treatment resistant depression. In other embodiments, a refractory individual may have responded to the initial treatment, but not succeed in entering remission from the treatment. In some embodiments, refractory individuals may have been unable to continue taking the medication due to intolerance of the medication including side effects such as, but not limited to, sexual dysfunction, weight gain, insomnia, dry mouth, constipation, nausea and vomiting, dizziness, memory loss, agitation, anxiety, sedation, headache, urinary retention, or abdominal pain. Unsatisfactory or failed responses may be determined by any means generally used, including patient self-reporting, clinical observation and depression rating scales.

Administration of pharmaceutical compositions comprising (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts will be effective to improve an individual's score on a depression rating scale such as, but not limited to, Montgomery Åsberg Depression Rating Scale (MADRS), the Hamilton Rating Scale for Depression (HAMD-17), the Clinical Global Impression-Severity Scale (CGI-S) and the Clinical Global Impression-Improvement Scale (CGI-I). In some embodiments, administration of the pharmaceutical compositions contemplated herein will be sufficient to place an individual into remission. Remission may be measured by any of a variety of ways, for example, remission from depression may be determined with a MADRS score of ≦12, HAMD-17 score of ≦7 or CGI-S score of ≦2.

In accordance with this invention, a dosage form has been developed for the sustained or extended release delivery of an active ingredient of (+)-1-(3,4-dichlorophenyl)-3-azabicyclo[3.1.0]hexane agents including (+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane in effective amounts to treat disorders affected by monoamine neurotransmitters, particularly depression, for a long period of time. In accordance with the invention, the active ingredient can be administered in an effective amount to provide sustained relief of depression by utilizing a dosage regimen of from about 25 mg, to about 200 mg, once or twice daily in an oral unit dosage form containing as its composition this amount of the active ingredient, 30% to 50% by weight of the composition of a pharmaceutically acceptable carrier, and from about 15% to 45% by weight of the composition of a hydroxypropyl methyl cellulose slow release matrix, with the carrier and the active ingredient dispersed in the slow release matrix.

The present invention may be understood more fully by reference to the detailed description and examples which are intended to exemplify non-limiting embodiments of the invention.

### BRIEF DESCRIPTION OF THE DRAWINGS

FIG. 1 is a graph showing a decrease in patients' scores on the Montgomery Åsberg Depression Rating Scale when treated with EB-1010 ((+)-1-(3,4-dichlorophenyl)-3-azabicyclo[3.1.0]hexane) in comparison to placebo in a six week double-blind study and one week post-treatment (modified intent-to-treat, n=56) (mixed-effects model repeated measures approach (MMRM) least square (LS) means).

FIG. 2 is a graph showing that treatment with EB-1010 ((+)-1-(3,4-dichlorophenyl)-3-azabicyclo[3.1.0]hexane) resulted in a decrease on the Hamilton Depression Rating Scale (HAM-D) in comparison to placebo in a six week double-blind study and one week post-treatment (modified intent-to-treat, n=56) (MMRM LS means).

FIG. 3 is a graph showing that treatment with EB-1010 ((+)-1-(3,4-dichlorophenyl)-3-azabicyclo[3.1.0]hexane) resulted in a decrease on the Clinical Global Impression-Improvement Scale (CGI-I) in a six week double-blind study and one week post-treatment indicating improvement in the condition of the patients in a six week double-blind study and one week post-treatment (modified intent-to-treat, n=56) (MMRM LS means).

FIG. 4 is a graph showing an improvement in the condition of patients treated with EB-1010 ((+)-1-(3,4-dichlorophenyl)-3-azabicyclo[3.1.0]hexane) in comparison to placebo in a six week double-blind study and one week post-treatment as determined using the Clinical Global Impression-Severity (CGI-S) scale. (Modified intent-to-treat, n=56) (MMRM LS means).

FIG. 5 is a graph showing that treatment with EB-1010 ((+)-1-(3,4-dichlorophenyl)-3-azabicyclo[3.1.0]hexane) resulted in significantly greater remission rates than treatment with placebo as measured by the Clinical Global Impressions-Severity (CGI-S) scale (Last Observation Carried Forward (LOCF)).

FIG. 6 is a graph showing that treatment with EB-1010 ((+)-1-(3,4-dichlorophenyl)-3-azabicyclo[3.1.0]hexane) resulted in statistically significant improvement on the adhedonia factor score of the MADRS compared to placebo in a six week double-blind study and one week post-treatment. (Modified intent-to-treat, n=56) (MMRM LS means).

FIG. 7 is a graph showing that Derogatis Interview for Sexual Functioning-Self Report (DISF-SR) scores stratified by low mean baseline scores versus high mean baseline scores and that there was no difference in those treated with EB-1010 ((+)-1-(3,4-dichlorophenyl)-3-azabicyclo[3.1.0]hexane) or placebo indicating that treatment with EB-1010 is not associated with the emergence of sexual dysfunction that is typically observed with serotonergic and serotonergic combination antidepressants (LOCF).

### DETAILED DESCRIPTION OF EXEMPLARY EMBODIMENTS

Described herein is an enantiomer of (±)-1-(3,4-dichlorophenyl)-3-azabicyclo[3.1.0]hexane which provides therapeutic efficacy in the treatment of conditions affected by monoamine neurotransmitters including, but not limited to, depression, anxiety, panic disorder, post-traumatic stress disorder, obsessive compulsive disorder, schizophrenia and allied disorders, obesity, tic disorders, addiction, attention deficit hyperactivity disorder (ADHD), Parkinson's disease, chronic pain and Alzheimer's disease. Further described herein are coordinate treatment methods and combined drug compositions, dosage forms, packages, and kits for preventing or treating conditions affected by monoamine neurotransmitters including, but not limited to, depression.

(±)-1-(3,4-dichlorophenyl)-3-azabicyclo[3.1.0]hexane is a triple reuptake inhibitor (TRI), or serotonin-norepinephrine-dopamine reuptake inhibitor (SNDRI). It was previously described in U.S. Pat. No. 4,435,419 to Epstein et al for use as an antidepressant. It possesses a desirable unbalanced triple monoamine uptake inhibition ratio, with highly potent serotonin reuptake inhibition and lesser norepinephrine and, particularly, dopamine reuptake inhibition in a ratio of ˜1:2:8, respectively (IC50 values of 12, 23, and 96 nM, respectively in human embryonic kidney (HEK) 293 cells expressing the corresponding human recombinant transporters for [3H]serotonin, [3H]norepinephrine, and [3H]dopamine). (Skolnick et al., 2003). There is preclinical evidence in support of the hypothesis that antidepressants that work by enhancing the synaptic availability of serotonin, norepinephrine, and dopamine may be superior to antidepressants that selectively affect only serotonin and/or norepinephrine reuptake. (Skolnick et al., 2003) The lesser dopamine reuptake inhibition is thought to be sufficient to confer a beneficial effect in the treatment of anhedonia (a core symptom presumably due to a mesocorticolimbic dopaminergic hypofunction in major depressive illness) and cognitive dysfunction, while avoiding undesirable effects thought to be triggered by excessive stimulation of dopamine systems, such as hypomania, nausea, insomnia or excessive pleasure seeking behaviors. Additionally, an unbalanced triple reuptake inhibitor may provide a lower side effect profile than a balanced triple reuptake inhibitor and allow for higher concentrations of an unbalanced inhibitor such as (+)-1-(3,4-dichlorophenyl)-3-azabicyclo[3.1.0]hexane to be used without incurring the dopaminergic and/or noradrenergic side effects frequently seen in the use of balanced triple reuptake inhibitors or unbalanced triple reuptake inhibitors that have different inhibition ratios.

Provided herein are compositions and methods using (+)-1-(3,4-dichlorophenyl)-3-azabicyclo[3.1.0]hexane as shown below, and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane, for the treatment of mammals, including humans, suffering from signs and symptoms of disorders generally treated with triple reuptake inhibitors including, but not limited to, depression, anxiety, panic disorder, post-traumatic stress disorder, obsessive compulsive disorder, schizophrenia and allied disorders, addiction, obesity, tic disorders, attention deficit hyperactivity disorder (ADHD), Parkinson's disease, chronic pain and Alzheimer's disease. (+)-1-(3,4-dichlorophenyl)-3-azabicyclo[3.1.0]hexane is particularly useful in treating depression in those who have been previously treated for a condition affected by monoamine neurotransmitters, specifically those who have failed an initial course of antidepressant therapy, such as selective serotonin reuptake inhibitor therapy.

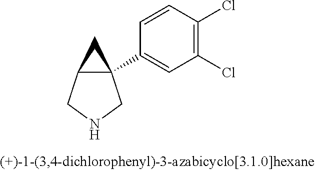 [SMILES: Clc1ccc([C@]23CNC[C@H]2C3)cc1Cl]

(+)-1-(3,4-dichlorophenyl)-3-azabicyclo[3.1.0]hexane may be prepared by any means generally used for preparing such a compound. For example, the (+) enantiomer may be synthesized by asymmetric synthesis using optically active reagents, substrates, catalysts or solvents, or by converting one enantiomer to the other by asymmetric transformation. An efficient means of preparing (+)-1-(3,4-dichlorophenyl)-3-azabicyclo[3.1.0]hexane is described in U.S. patent application Ser. No. 11/740,667, incorporated herein by reference in its entirety. Additional exemplary means of preparing (+)-1-(3,4-dichlorophenyl)-3-azabicyclo[3.1.0]hexane may be found, for example, in U.S. patent application Ser. Nos. 10/920,748, 11/205,956; 12/208,284; 12/428,399, WO20040466457, WO2007127396, WO02066427, WO2006023659, and U.S. Pat. No. 6,372,919, each of which is incorporated herein by reference in its entirety.

Alternatively, (+)-1-(3,4-dichlorophenyl)-3-azabicyclo[3.1.0]hexane may be isolated from (±)-1-(3,4-dichlorophenyl)-3-azabicyclo[3.1.0]hexane by any means generally used. Methods for preparing (±)-1-(3,4-dichlorophenyl)-3-azabicyclo[3.1.0]hexane may be found, for example, in U.S. Pat. No. 4,435,419 and U.S. patent application Ser. Nos. 10/920,748, 11/205,956; 12/208,284; 12/428,399 each of which is incorporated herein by reference in their entirety. The enantiomers of (±)-1-(3,4-dichlorophenyl)-3-azabicyclo[3.1.0]hexane may be separated by any means generally used to separate enantiomeric forms including, but not limited to, crystallization, the use of chiral acids, oxidation of corresponding chiral amino alcohols (Berrang, B. D., et al., 1982), reduction with BH₃-THF, liquid chromatography, gas-liquid chromatography, chiral columns, high performance liquid chromatography (HPLC), the use of an ovomucoid column, electrokinetic chromatography, selective reaction of one reaction of one enantiomer with an enantiomer-specific reagent, and the use of highly sulfated cyclodextrins among others. As used herein, the term “substantially pure (+)-1-(3,4-dichlorophenyl)-3-azabicyclo[3.1.0]hexane” or “enantiomerically pure (+)-1-(3,4-dichlorophenyl)-3-azabicyclo[3.1.0]hexane” means that the compositions contain more (+)-1-(3,4-dichlorophenyl)-3-azabicyclo[3.1.0]hexane than (−)-1-(3,4-dichlorophenyl)-3-azabicyclo[3.1.0]hexane. Specifically, the compositions refer to an enantiomeric excess greater than 80%, preferably greater than 90%, more preferably greater than 95%, and most preferably greater than 98% of the (+)-1-(3,4-dichlorophenyl)-3-azabicyclo[3.1.0]hexane as determined by configuration and/or optical activity. Typically, the compositions contain no more than about 5% w/w of the corresponding (−) enantiomer, more preferably no more than about 2%, more preferably no more than about 1% w/w of the corresponding (−) enantiomer of (+)-1-(3,4-dichlorophenyl)-3-azabicyclo[3.1.0]hexane.

(+)-1-(3,4-dichlorophenyl)-3-azabicyclo[3.1.0]hexane is polymorphic. The present invention comprises use of one or more polymorphic forms of (+)-1-(3,4-dichlorophenyl)-3-azabicyclo[3.1.0]hexane, specifically forms A, B and C as disclosed in U.S. patent application Ser. Nos. 11/205,956, 12/208,284 and 12/428,399 incorporated herein by reference in their entirety.

Polymorph form A may be characterized as the hemi-hydrate of acid addition salts of (+)-1-(3,4-dichlorophenyl)-3-azabicyclo[3.1.0]hexane. The polymorphs of acid addition salts of (+)-1-(3,4-dichlorophenyl)-3-azabicyclo[3.1.0]hexane may be characterized by their X-ray powder diffraction patterns (XRPD) and/or their Raman spectroscopy peaks. A Bragg-Brentano instrument, which includes the Shimadzu system, used for the X-ray powder diffraction pattern measurements reported herein, gives a systematic peak shift (all peaks can be shifted at a given “°2θ” angle) which result from sample preparation errors as described in Chen et al.; J Pharmaceutical and Biomedical Analysis, 2001; 26, 63. Therefore, any “°2θ” angle reading of a peak value is subject to an error of about (±) 0.2°.

The following Table 1 shows the values for the relative intensities for peaks of the X-ray powder diffraction pattern of purified polymorph form A of the hydrochloride salt of (+)-1-(3,4-dichlorophenyl)-3-azabicyclo[3.1.0]hexane having a crystal size of from about 10 to 40 microns. With respect to the percent value of relative intensity (I/lo) given in Table 1, Io represents the value of the maximum peak determined by XRPD for the sample for all “°2θ” angles and I represents the value for the intensity of a peak measured at a given “°2θ” angle”. The angle “°2θ” is a diffraction angle which is the angle between the incident X-rays and the diffracted X-rays.

TABLE 1
XRPD Peaks (°2θ) and Relative Intensities
(l/lo) for Polymorph Form A
Form A

| °2θ | l/lo |
|---|---|
| 4.55 | 25 |
| 9.10 | 15 |
| 13.65 | 6 |
| 17.14 | 60 |
| 17.85 | 11 |
| 18.24 | 23 |
| 18.49 | 14 |
| 19.27 | 14 |
| 19.62 | 22 |
| 21.74 | 15 |
| 21.96 | 100 |
| 22.24 | 12 |
| 23.01 | 7 |
| 24.52 | 43 |
| 24.79 | 10 |
| 26.74 | 52 |
| 27.44 | 11 |
| 27.63 | 17 |
| 28.36 | 16 |
| 28.48 | 26 |
| 29.00 | 14 |
| 29.20 | 19 |
| 29.40 | 27 |
| 29.57 | 27 |
| 30.24 | 18 |
| 31.01 | 13 |
| 31.62 | 17 |
| 32.20 | 24 |
| 32.93 | 12 |
| 33.42 | 9 |
| 34.24 | 6 |
| 35.08 | 15 |
| 35.65 | 16 |
| 36.31 | 14 |
| 37.11 | 26 |
| 37.78 | 9 |
| 39.85 | 9 |

The following Table 2 shows the relative intensities for peaks of the X-ray powder diffraction pattern of purified polymorph form B of the hydrochloride salt of (+)-1-(3,4-dichlorophenyl)-3-azabicyclo[3.1.0]hexane having a crystal size of from about 10 to 40 microns.

TABLE 2
XRPD Peaks (°2θ) and Relative Intensities
(l/lo) for Polymorph Form B
Form B

| °2θ | l/lo |
|---|---|
| 10.50 | 6 |
| 13.34 | 12 |
| 15.58 | 42 |
| 17.12 | 6 |
| 17.36 | 8 |
| 17.52 | 26 |
| 18.21 | 11 |
| 20.40 | 7 |
| 21.35 | 97 |
| 21.61 | 17 |
| 21.93 | 11 |
| 22.64 | 6 |
| 23.04 | 79 |
| 24.09 | 6 |
| 24.52 | 14 |
| 25.43 | 96 |
| 26.24 | 53 |
| 26.36 | 73 |
| 26.75 | 11 |
| 26.88 | 7 |
| 27.44 | 6 |
| 27.94 | 12 |
| 28.36 | 20 |
| 28.54 | 30 |
| 29.39 | 10 |
| 29.72 | 9 |
| 30.07 | 7 |
| 30.58 | 8 |
| 30.72 | 100 |
| 31.07 | 14 |
| 31.38 | 12 |
| 31.55 | 7 |
| 31.78 | 12 |
| 32.14 | 10 |
| 32.31 | 7 |
| 32.80 | 7 |
| 32.95 | 6 |
| 33.45 | 44 |
| 33.74 | 12 |
| 35.25 | 10 |
| 35.40 | 12 |
| 35.58 | 9 |
| 36.75 | 8 |
| 37.55 | 18 |
| 39.01 | 15 |
| 39.22 | 7 |
| 39.37 | 7 |
| 39.86 | 11 |

The following Table 3 shows the values of the relative intensities of the peaks of the X-ray powder diffraction pattern of purified polymorph form C of the hydrochloride salt of (+)-1-(3,4-dichlorophenyl)-3-azabicyclo[3.1.0]hexane having a crystal size of from about 10 to 40 microns.

TABLE 3
XRPD Peaks (°2θ) and Relative Intensities
(I/lo) for Polymorph Form C
Form C

| °2θ | l/lo |
|---|---|
| 5.46 | 6 |
| 5.66 | 20 |
| 6.37 | 6 |
| 7.26 | 6 |
| 8.75 | 6 |
| 13.34 | 25 |
| 13.94 | 11 |
| 15.65 | 7 |
| 16.26 | 7 |
| 17.01 | 8 |
| 17.38 | 9 |
| 17.64 | 83 |
| 17.92 | 15 |
| 18.23 | 40 |
| 19.08 | 7 |
| 19.38 | 46 |
| 19.86 | 20 |
| 20.07 | 100 |
| 21.16 | 17 |
| 21.32 | 94 |
| 21.64 | 37 |
| 22.42 | 25 |
| 22.70 | 12 |
| 22.97 | 70 |
| 23.31 | 6 |
| 24.09 | 15 |
| 24.86 | 94 |
| 25.24 | 32 |
| 25.38 | 49 |
| 26.12 | 13 |
| 26.32 | 90 |
| 26.87 | 18 |
| 27.21 | 39 |
| 27.90 | 54 |
| 28.14 | 8 |
| 28.56 | 32 |
| 28.74 | 17 |
| 29.20 | 6 |
| 29.72 | 6 |
| 29.92 | 26 |
| 30.54 | 13 |
| 30.72 | 19 |
| 30.96 | 31 |
| 31.42 | 7 |
| 31.68 | 11 |
| 31.80 | 15 |
| 31.97 | 6 |
| 32.43 | 21 |
| 33.26 | 12 |
| 33.40 | 15 |
| 33.64 | 25 |
| 33.84 | 18 |
| 34.11 | 15 |
| 34.70 | 11 |
| 35.07 | 8 |
| 35.64 | 11 |
| 35.91 | 8 |
| 36.09 | 21 |
| 37.80 | 12 |
| 38.06 | 6 |
| 38.17 | 6 |
| 39.04 | 6 |
| 39.23 | 8 |
| 39.77 | 7 |

There are key major peaks at given angles in these X-ray powder diffraction patterns which are unique to each given polymorph form. These peaks are present in the XRPD patterns of each of the polymorph forms having a crystal size of about 10 to 40 microns. Any of these major peaks, either alone or in any distinguishing combination, are sufficient to distinguish one of the polymorph forms from the other two polymorph forms. For polymorph form A, the “°2θ” angles of these major peaks which characterize polymorph form A, subject to the error set forth above, are as follows: 17.14; 19.62; 21.96; 24.52; and 26.74. For polymorph form B, the “°2θ” angles of these major peaks which characterize polymorph form B, subject to the error set forth above, are as follows: 15.58; 17.52; 21.35; 23.04; 25.43; and 30.72. For polymorph form C, the “°2θ” angles of these major peaks which characterize polymorph form C, subject to the error set forth above, are as follows: 13.34; 17.64; 20.07; 21.32; 22.97; 24.86; 26.32; and 27.90. Any of these major peaks, either alone or in any distinguishing combination, are sufficient to distinguish a polymorph from the other polymorph forms.

Another method of characterizing the three polymorphs of (+)-1-(3,4-dichlorophenyl)-3-azabicyclo[3.1.0]hexane is through Raman spectroscopy. The procedure for carrying out Raman Spectroscopy is described on pages 260-275 of Skoog and West, Principles of Instrumental Analysis (2nd Ed.); Saunders College, Philadelphia (1980).

The Raman spectra peak positions in wavenumbers (cm⁻¹) for polymorph form A, B and C of the hydrochloride salt of (+)-1-(3,4-dichlorophenyl)-3-azabicyclo[3.1.0]hexane are given in Table 4, below.

TABLE 4
Raman Peak Listing for Polymorph Form A, B and C
(peaks>400 cm⁻¹)
Peak Positions In Wavenumbers (cm⁻¹)

| | | |
|---|---|---|
| Form A | Form B | Form C |

| | | | | | |
|---|---|---|---|---|---|
| 436 | 418 | 441 | 1246 | 1245 | 1135 |
| 479 | 446 | 474 | 1266 | 1278 | 1189 |
| 534 | 478 | 532 | 1279 | 1309 | 1229 |
| 549 | 533 | 648 | 1309 | 1343 | 1274 |
| 646 | 648 | 674 | 1343 | 1380 | 1309 |
| 691 | 676 | 690 | 1398 | 1398 | 1338 |
| 680 | 686 | 767 | 1456 | 1456 | 1366 |
| 762 | 767 | 811 | 1471 | 1483 | 1393 |
| 812 | 825 | 826 | 1557 | 1557 | 1453 |
| 836 | 852 | 856 | 1595 | 1593 | 1484 |
| 892 | 895 | 895 | 2900 | 2895 | 1557 |
| 921 | 964 | 970 | 2966 | 2963 | 1597 |
| 959 | 979 | 1031 | 2992 | 2993 | 2890 |
| 982 | 1031 | 1059 | 3048 | 3027 | 2969 |
| 998 | 1054 | 1094 | 3070 | 3066 | 2982 |
| 1030 | 1070 | 1122 | | | 3017 |
| 1056 | 1099 | 1137 | | | 3046 |
| 1099 | 1136 | 1189 | | | 3064 |
| 1122 | 1189 | 1228 | | | |

Table 4 provides the complete patterns of the Raman peak positions with respect to the hydrochloride salts of polymorph forms A, B and C respectively. However, there are certain key peaks within these patterns which are unique to each of the hydrochloride salts of these polymorphs. Any of these key peaks, either alone or in any distinguishing combination, are sufficient to distinguish one of the polymorph forms from the other two polymorph forms. These peak positions, expressed in wavenumbers (cm⁻¹) for the hydrochloride salt of polymorph form A are: 762; 636; 921; 959; 1393; 1597; 2890; 2982; and 3064. The characterizing peak positions expressed in wavenumbers (cm⁻¹) for the hydrochloride salt of polymorph form B are: 1245; 1380; 2963; 2993; 3027; and 3066. The characterizing peak positions expressed in wavenumbers (cm⁻¹) for the hydrochloride salt of polymorph form C are: 1059; 1094; 1266; 1343; 1595; 2900; 2966; and 3070. Any of these key peaks, either alone or in any distinguishing combination, are sufficient to distinguish each polymorph form from the other two polymorph forms.

Polymorph forms A, B and C of (+)-1-(3,4-dichlorophenyl)-3-azabicyclo[3.1.0]hexane, particularly as hydrochloride acid addition salts, can each be prepared substantially free of its other enantiomeric, geometric and polymorphic isomeric forms through re-crystallization of a mixture of the A and B polymorph forms produced in accordance with prior art procedures. Depending upon the particular solvent, conditions and concentrations of materials utilized to re-crystallize the mixture of polymorph forms A and B, one can selectively produce the desired polymorph form of (+)-1-(3,4-dichlorophenyl)-3-azabicyclo[3.1.0]hexane, substantially free of its other enantiomeric, geometric and polymorphic isomers. The term “substantially free” of its other enantiomeric, geometric and polymorphic isomeric forms designates that the crystalline material is at least about 95% by weight pure in that it contains no more than about 5% w/w of its other enantiomeric, geometric and polymorphic isomeric forms.

Any means generally used to separate polymorphs may be used. For example, in preparing polymorph forms A and B substantially free of other polymorph forms, crystallization from a mixture of A and B may be utilized. However, the crystallization technique with regard to producing each of these polymorph forms substantially free of other polymorph forms is different. In preparing polymorph form A, which is the hemi-hydrate of the acid addition salt of (+)-1-(3,4-dichlorophenyl)-3-azabicyclo[3.1.0]hexane, it is best to utilize a solvent medium to dissolve a solid containing polymorph form A such as a mixture of polymorph forms A and B in an organic solvent which contains water. The preferred organic solvents that can be utilized in this procedure include lower alkanol solvents such as methanol, butanol, ethanol or isopropanol as well as other solvents such as acetone, dichloromethane and tetrahydrofuran.

Polymorph form B is the anhydrous form of the acid addition salt of (+)-1-(3,4-dichlorophenyl)-3-azabicyclo[3.1.0]hexane. Polymorph form B of the acid addition salt of (+)-1-(3,4-dichlorophenyl)-3-azabicyclo[3.1.0]hexane can be prepared from a solid containing polymorph form A or a mixture of polymorph forms A and B by dissolving the polymorph form A or the mixture of polymorph forms A and B, preferably as the hydrochloride salt, utilizing anhydrous conditions.

Polymorph form C can be prepared from either polymorph form A or polymorph form B or mixtures thereof. Polymorph form C is prepared by extensive heating of either polymorph form A or polymorph form B, or mixtures thereof, at temperatures of at least 50° C., preferably from 60° C. to 80° C. Heating can be continued until polymorph form C substantially free of other polymorph forms is formed.

The techniques set forth above also allow for the preparation of mixtures of the individual polymorph forms of the acid addition salt of (+)-1-(3,4-dichlorophenyl)-3-azabicyclo[3.1.0]hexane containing specific amounts of each of the polymorphs. In particular, mixtures of polymorph form A and either polymorph form B or polymorph form C; polymorph form B and polymorph form C; and polymorph form A, polymorph form B and polymorph form C can be readily prepared with the desired amounts of each of the polymorphs. Using the techniques set forth above, mixtures containing specific percentages of the individual polymorphic forms of the acid addition salt of (+)-1-(3,4-dichlorophenyl)-3-azabicyclo[3.1.0]hexane can be obtained. For example, mixtures containing from about 10% to about 10-20%, 20-35%, 35-50%, 50-70%, 70-85%, 85-95% and up to 95-99% or greater (by weight) of polymorph form A, with the remainder of the mixture being either or both polymorph form B and polymorph form C, can be prepared. As another example, mixtures containing from about 10% to about 10-20%, 20-35%, 35-50%, 50-70%, 70-85%, 85-95% and up to 95-99% or greater (by weight) of polymorph form B, with the remainder of the mixture being either or both polymorph form A and polymorph form C, can be prepared. As a further example, mixtures containing from about 10% to about 10-20%, 20-35%, 35-50%, 50-70%, 70-85%, 85-95% and up to 95-99% or greater (by weight) of polymorph form C, with the remainder of the mixture being either or both polymorph form A and polymorph form B, can be prepared.

Additionally, many pharmacologically active organic compounds regularly crystallize incorporating second, foreign molecules, especially solvent molecules, into the crystal structure of the principal pharmacologically active compound to form pseudopolymorphs. When the second molecule is a solvent molecule, the pseudopolymorphs can also be referred to as solvates. All of these additional forms of (+)-1-(3,4-dichlorophenyl)-3-azabicyclo[3.1.0]hexane are likewise contemplated for use within the present invention.

The polymorph forms A, B and C of the present invention can be prepared as acid addition salts formed from an acid and the basic nitrogen group of (+)-1-(3,4-dichlorophenyl)-3-azabicyclo[3.1.0]hexane. Suitable acid addition salts are formed from acids, which form non-toxic salts, examples of which are hydrochloride, hydrobromide, hydroiodide, sulphate, hydrogen sulphate, nitrate, phosphate, and hydrogen phosphate. Examples of pharmaceutically acceptable addition salts include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like. The hydrochloride salt formed with hydrochloric acid is an exemplary useful salt.

As disclosed herein, (+)-1-(3,4-dichlorophenyl)-3-azabicyclo[3.1.0]hexane agents ((+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) are effective in treating a variety of conditions including, but not limited to, depression, anxiety, panic disorder, post-traumatic stress disorder, obsessive compulsive disorder, schizophrenia and allied disorders, addiction, obesity, tic disorders, Parkinson's disease, ADHD, chronic pain and Alzheimer's disease. Within related aspects of the invention, combinatorial formulations are provided that use substantially pure (+)-1-(3,4-dichlorophenyl)-3-azabicyclo[3.1.0]hexane, or pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane alone or in combination with other psychotherapeutic drugs to modulate, prevent, alleviate, ameliorate, reduce or treat symptoms or conditions influenced by monoamine neurotransmitters or biogenic amines. Subjects amenable to treatment according to the invention include mammalian subjects, including humans, suffering from or at risk for any of a variety of conditions including, but not limited to, depression, anxiety, panic disorder, post-traumatic stress disorder, obsessive compulsive disorder, schizophrenia and allied disorders, obesity, tic disorders, addiction, ADHD, Parkinson's disease, chronic pain and Alzheimer's disease.

(+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be administered alone or in combination with one or more other psychotherapeutic drugs including, but not limited to, drugs from the general classes of anti-convulsant, mood-stabilizing, anti-psychotic, anxiolytic, benzodiazepines, calcium channel blockers, and antidepressants. (See, e.g., R J. Baldessarini in Goodman & Gilman's The Pharmacological Basis of Therapeutics, 11th Edition, Chapters 17 and 18, McGraw-Hill, 2005 for a review). Additionally, (+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be administered in combination with an anti-inflammatory.

Within the coordinate administration methods of the invention, the secondary therapeutic and/or psychotherapeutic drug is administered concurrently or sequentially with (+)-1-(3,4-dichlorophenyl)-3-azabicyclo[3.1.0]hexane, or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane to treat or prevent one or more symptoms of the targeted disorder. When administered simultaneously, the additional therapeutic and/or psychotherapeutic agent and (+)-1-(3,4-dichlorophenyl)-3-azabicyclo[3.1.0]hexane or a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane may be combined in a single composition or combined dosage form. Alternatively, the combinatorially effective additional therapeutic and/or psychotherapeutic drug and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) may be administered at the same time in separate dosage forms. When the coordinate administration is conducted simultaneously or sequentially, the additional therapeutic and/or psychotherapeutic agent and (+)-1-(3,4-dichlorophenyl)-3-azabicyclo[3.1.0]hexane agent may each exert biological activities and therapeutic effects over different time periods, although a distinguishing aspect of all coordinate treatment methods of the invention is that treated subjects exhibit positive therapeutic benefits.

Administration of (+)-1-(3,4-dichlorophenyl)-3-azabicyclo[3.1.0]hexane, a pharmaceutically acceptable active salt, polymorph, glycosylated derivative, metabolite, solvate, hydrate, and/or prodrug of (+)-1-(3,4-dichlorophenyl)-3-azabicyclo[3.1.0]hexane or the coordinate treatment method or combinatorial drug composition of the invention to suitable subjects will yield a reduction in one or more target symptom(s) associated with the selected disorder or development of the disorder by at least 2%, 5%, 10%, 20%, 30%, 50% or greater, up to a 75-90%, or 95% or greater, compared to placebo-treated or other suitable control subjects. Comparable levels of efficacy are contemplated for the entire range of disorders described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

As used herein, the terms “prevention” and “preventing,” when referring to a disorder or symptom, refers to a reduction in the risk or likelihood that a mammalian subject will develop said disorder, symptom, condition, or indicator after treatment according to the invention, or a reduction in the risk or likelihood that a mammalian subject will exhibit a recurrence or relapse of said disorder, symptom, condition, or indicator once a subject has been treated according to the invention and cured or restored to a normal state (e.g., placed in remission from a targeted disorder). As used herein, the terms “treatment” or “treating,” when referring to the targeted disorder, refers to inhibiting or reducing the progression, nature, or severity of the subject condition or delaying the onset of the condition.

In accordance with the invention, compounds disclosed herein, optionally formulated with additional ingredients in a pharmaceutically acceptable composition, are administered to mammalian subjects, for example a human patient, to treat or prevent one or more symptom(s) of a disorder alleviated by inhibiting dopamine reuptake, and/or norepinephrine reuptake, and/or serotonin reuptake. In certain embodiments, “treatment” or “treating” refers to amelioration of one or more symptom(s) of a disorder, whereby the symptom(s) is/are alleviated by inhibiting dopamine and/or norepinephrine and/or serotonin reuptake. In other embodiments, “treatment” or “treating” refers to an amelioration of at least one measurable physical parameter associated with a disorder. In yet another embodiment, “treatment” or “treating” refers to inhibiting or reducing the progression or severity of a disorder (or one or more symptom(s) thereof) alleviated by inhibiting dopamine and/or norepinephrine and/or serotonin reuptake, e.g., as discerned based on physical, physiological, and/or psychological parameters. In additional embodiments, “treatment” or “treating” refers to delaying the onset of a disorder (or one or more symptom(s) thereof) alleviated by inhibiting dopamine and/or norepinephrine and/or serotonin reuptake.

An “effective amount,” “therapeutic amount,” “therapeutically effective amount,” or “effective dose” of (+)-1-(3,4-dichlorophenyl)-3-azabicyclo[3.1.0]hexane agent (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) and/or an additional psychotherapeutic agent as used herein means an effective amount or dose of the active compound as described herein sufficient to elicit a desired pharmacological or therapeutic effect in a human subject. In the case of antidepressant therapeutic agents, these terms most often refer to a measurable, statistically significant reduction in an occurrence, frequency, or severity of one or more symptom(s) of a specified disorder, including any combination of neurological and/or psychological symptoms, diseases, or conditions, associated with or caused by the targeted disorder and/or reduction in the development of depression in a target population.

Therapeutic efficacy can alternatively be demonstrated by a decrease in the frequency or severity of symptoms associated with the treated condition or disorder, or by altering the nature, occurrence, recurrence, or duration of symptoms associated with the treated condition or disorder. In this context, “effective amounts,” “therapeutic amounts,” “therapeutically effective amounts,” and “effective doses” of additional psychotherapeutic drugs and (+)-1-(3,4-dichlorophenyl)-3-azabicyclo[3.1.0]hexane agents (including pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane) within the invention can be readily determined by ordinarily skilled artisans following the teachings of this disclosure and employing tools and methods generally known in the art, often based on routine clinical or patient-specific factors.

Efficacy of the coordinate treatment methods and drug compositions of the invention will often be determined by use of conventional patient surveys or clinical scales to measure clinical indices of disorders in subjects. The methods and compositions of the invention will yield a reduction in one or more scores or selected values generated from such surveys or scales completed by test subjects (indicating for example an incidence or severity of a selected disorder), by at least 5%, 10%, 20%, 30%, 50% or greater, up to a 75-90%, or 95% compared to correlative scores or values observed for control subjects treated with placebo or other suitable control treatment. In at risk populations, the methods and compositions of the invention will yield a stable or minimally variable change in one or more scores or selected values generated from such surveys or scales completed by test subjects. More detailed data regarding efficacy of the methods and compositions of the invention can be determined using alternative clinical trial designs.

Useful patient surveys and clinical scales for comparative measurement of clinical indices of psychiatric disorders in subjects treated using the methods and compositions of the invention can include any of a variety of widely used and well known surveys and clinical scales. Among these useful tools are the Mini International Neuropsychiatric Interview© (MINI) (Sheehan et al., 1998); Clinical Global Impression scale (CGI) (Guy, W., ECDEU Assessment Manual for Psychopharmacology, DHEW Publication No. (ADM) 76-338, rev. 1976); Clinical Global Impression Severity of Illness (CGI-S) (Guy, 1976); Clinical Global Impression Improvement (CGI-I) (Guy, et al. 1976); Beck Depression Inventory (BDI) (Beck, 2006); Revised Hamilton Rating Scale for Depression (RHRSD) (Warren, 1994); Major Depressive Inventory (MDI) (Olsen et al. 2003); and Children's Depression Index (CDI) (Kovacs, et al. 1981); Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Åsberg Depression Rating Scale® (MADRS) (Montgomery and Åsberg, 1979); Beck Scale for Suicide Ideation® (BSS) (Beck and Steer, 1991 Columbia-Suicide Severity Rating Scale© (C-SSRS) or Columbia Classification Algorithm of Suicide Assessment© (C CASA) (Posner, K., et al., 2007); Sheehan-Suicidality Tracking Scale© (S-SST) (Coric et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983); and the HAM-D scale for depression (Hamilton, 1960).

The methods and compositions of the invention will yield a reduction in one or more scores or values generated from these clinical surveys (using any single scale or survey, or any combination of one or more of the surveys described above) by at least 10%, 20%, 30%, 50% or greater, up to a 75-90%, or 95% compared to correlative scores or values observed for control subjects treated with placebo or other suitable control treatment. In prophylactic treatment, the methods and compositions of the invention will yield a stabilization or diminished change in the scores or values generated from these clinical surveys.

In some embodiments, administration of the pharmaceutical compositions contemplated herein will be sufficient to place an individual into remission for a condition, specifically depression. Remission from depression may be measured by any of a variety of ways, for example with patient surveys and clinical scales. An indication of remission, for example would be scores on the MADRS ≦12, HAMD-17≦7 or CGI-S≦2.

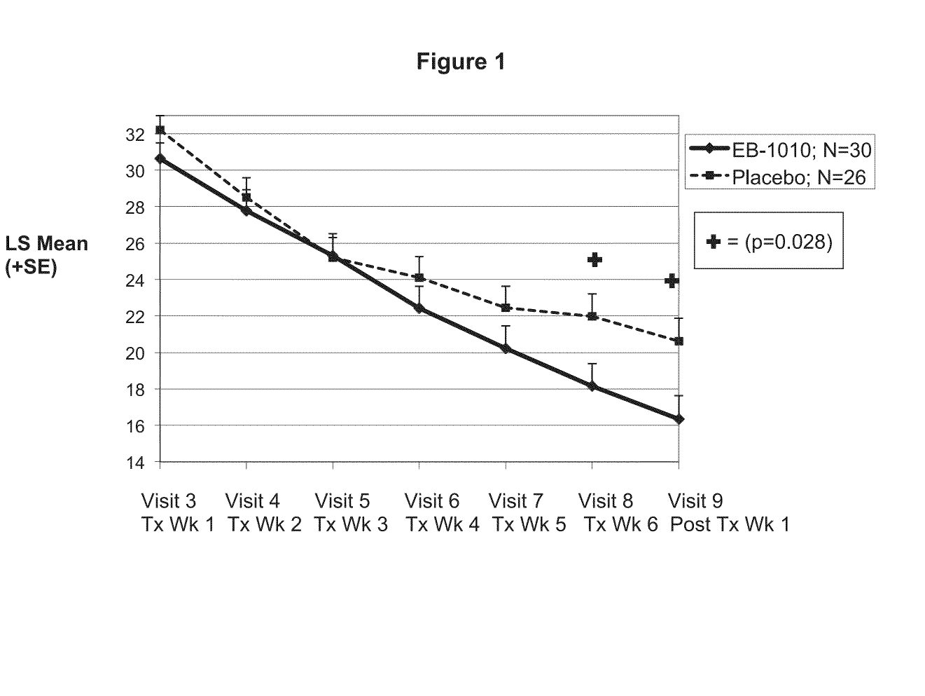

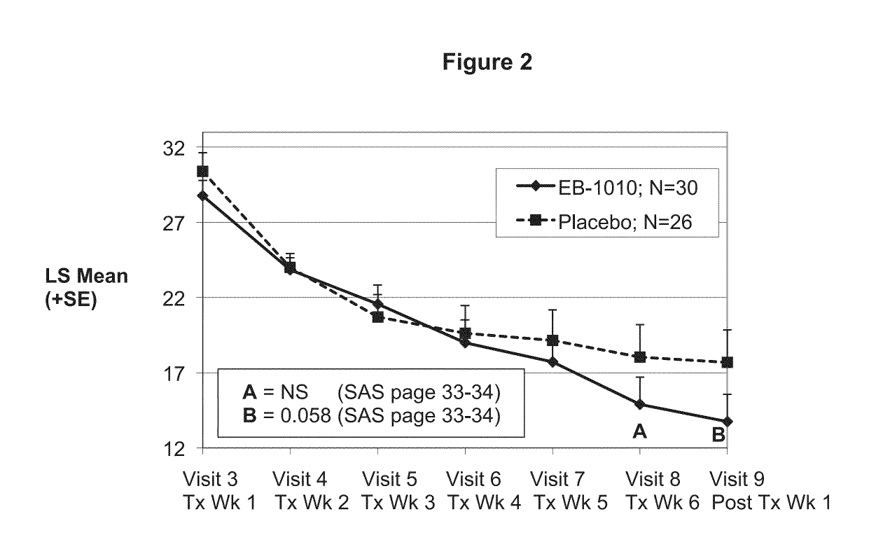

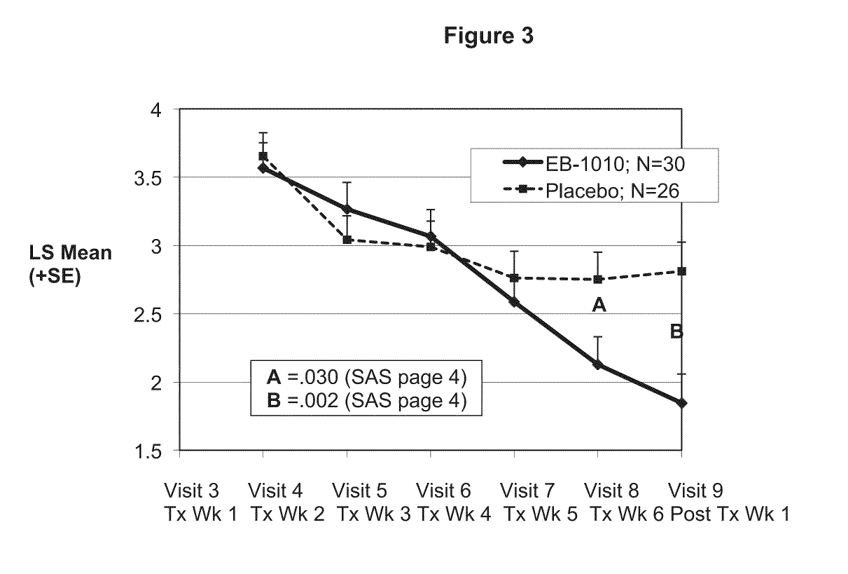

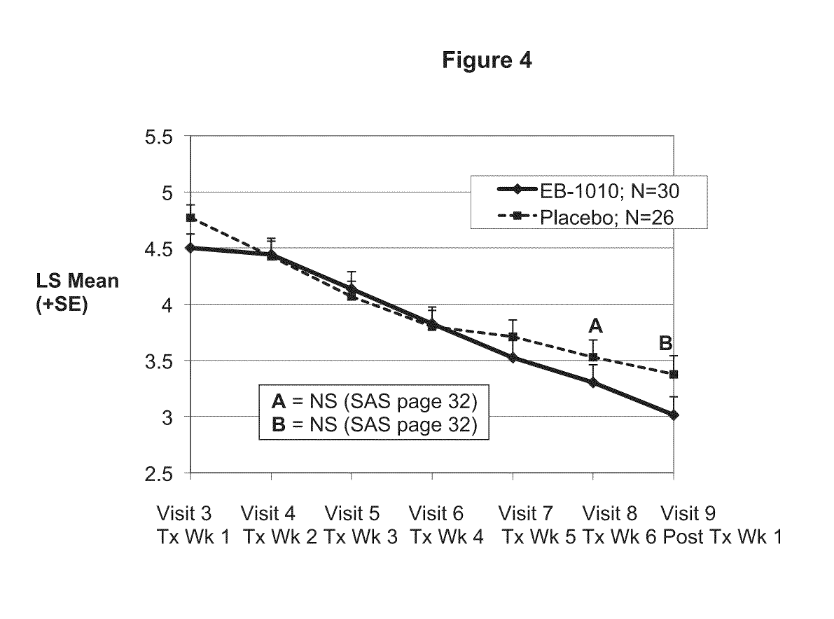

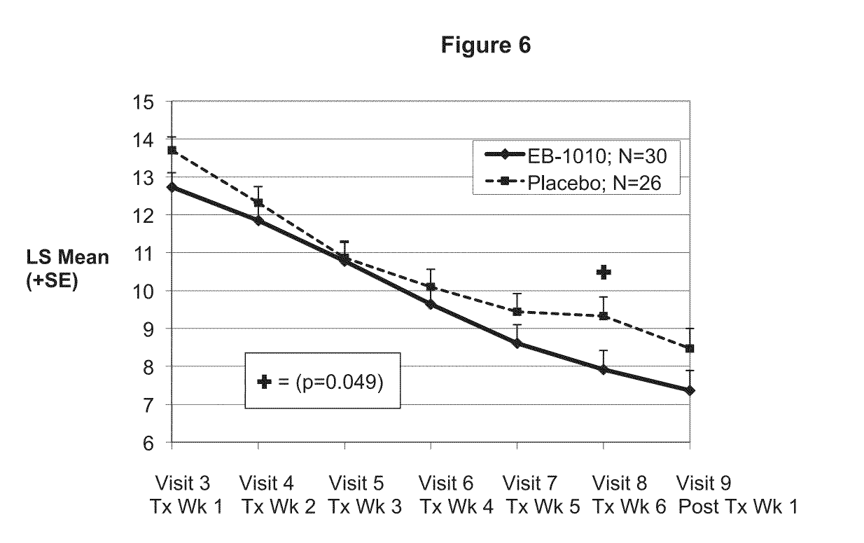

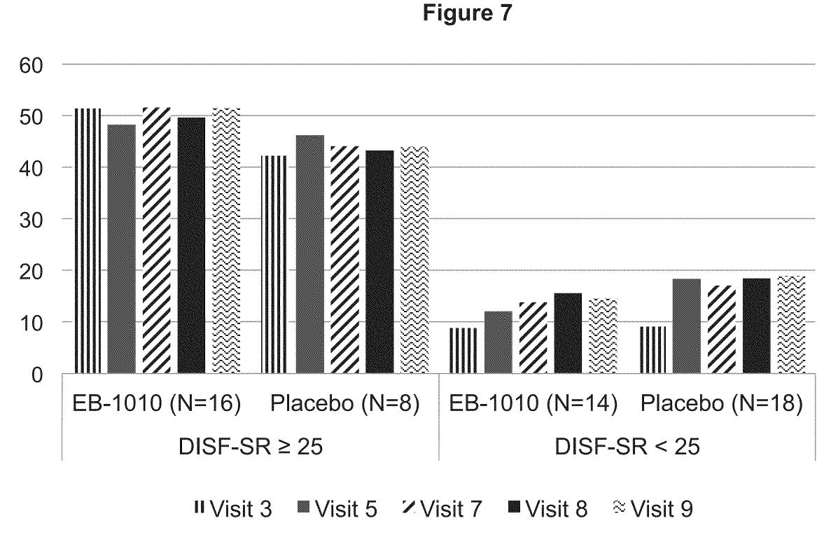

As shown in the figures above and examples below, administration of (+)-1-(3,4-dichlorophenyl)-3-azabicyclo[3.1.0]hexane in comparison to placebo in a six-week double blind study significantly decreased the depression levels in patients as measured using the Montgomery-Åsberg Depression rating scale (FIG. 1, data analyzed using the mixed model for repeated measures least square means (MMRM LS)), the Hamilton Depression Rating Scale (FIG. 2, data analyzed using the mixed model for repeated measures LS means), Clinical Global Impression Improvement (CGI-I) (FIG. 3, data analyzed using the mixed model for repeated measures LS means (MMRM LS), and the Clinical Global Impression Severity of Illness (CGI-S) (FIG. 4, data analyzed using the mixed model for repeated measures LS means). Treatment with (+)-1-(3,4-dichlorophenyl)-3-azabicyclo[3.1.0]hexane was also associated with statistically significant improvement on the anhedonia factor score of the MADRS compared to placebo (FIG. 6, data analyzed using the mixed model for repeated measures LS means (MMRM LS). Additionally, treatment with (+)-1-(3,4-dichlorophenyl)-3-azabicyclo[3.1.0]hexane showed no difference in comparison with placebo in evaluation of sexual dysfunction (FIG. 7, data analyzed using the last observation carried forward method (LOCF), indicating that (+)-1-(3,4-dichlorophenyl)-3-azabicyclo[3.1.0]hexane is not associated with emergence of sexual dysfunction. These results demonstrate surprising efficacy in comparison to other triple reuptake inhibitors. For example, SEP-225289, a triple reuptake inhibitor that underwent. Phase II clinical testing by Sepracor, did not meet the primary efficacy endpoint compared to placebo, which was a reduction in symptoms of depression following eight weeks of treatment, as assessed using the clinician-rated, 17-item HAM-D scale (Sepracor Press Release, Jul. 1, 2009). Similarly, GSK372475, a balanced triple reuptake inhibitor in development by GlaxoSmithKline, also failed to demonstrate a significant benefit in comparison to placebo. (Graff, Ole et al. 2009).

Additionally, the unbalanced reuptake inhibition profile of (+)-1-(3,4-dichlorophenyl)-3-azabicyclo[3.1.0]hexane allows for higher doses of (+)-1-(3,4-dichlorophenyl)-3-azabicyclo[3.1.0]hexane to be used without incurring the side effects that limit the effectiveness of balanced triple reuptake inhibitors such as GSK372475. In contrast to GSK372475, (+)-1-(3,4-dichlorophenyl)-3-azabicyclo[3.1.0]hexane is well tolerated and has a similar adverse event profile as placebo. (See, Example 1× and Graff, et al. 2009). H-1-(3,4-dichlorophenyl)-3-azabicyclo[3.1.0]hexane use also did not lead to the noradrenergic side effects such as significantly elevated heart rate and increased systolic and diastolic blood pressure seen with GSK37425 (See Tables 11 and 12 and Graff, 2009) or dopaminergic side effects such as nausea, vomiting, and hypomania.

The SEP-22589 inhibition profile for 5-HT, NE and DA (IC₅₀'s, SEP-289: 15, 4 and 3 nM (Schrieber, 2009)) is about equipotent for norepinephrine and dopamine reuptake inhibition and less potent for serotonin reuptake inhibition, leading to higher rates of noradrenergic or dopaminergic side effects than similar anti-depressant effective amounts of (+)-1-(3,4-dichlorophenyl)-3-azabicyclo[3.1.0]hexane.

The use of (+)-1-(3,4-dichlorophenyl)-3-azabicyclo[3.1.0]hexane will have substantially fewer dopaminergic or noradrenergic side effects than use of similar doses of balanced triple reuptake inhibitors. The use of substantially pure (+)-1-(3,4-dichlorophenyl)-3-azabicyclo[3.1.0]hexane will reduce adverse effects including side effects by 1%, 3%, 10%, 20%, 30%, 50% or greater, up to a 75%, 80%, 90%, or 95% or greater over use of a balanced triple reuptake inhibitor. Additionally, the use of (+)-1-(3,4-dichlorophenyl)-3-azabicyclo[3.1.0]hexane will have fewer dopaminergic or noradrenergic side effects than triple reuptake inhibitors with higher rates of inhibition for dopamine or noradrenaline reuptake. Thus, the use of substantially pure (+)-1-(3,4-dichlorophenyl)-3-azabicyclo[3.1.0]hexane will allow relatively greater reuptake inhibition of the 5-HT (serotonin) transporter, less of the NE (norepinephrine) transporter and even less of the DA (dopamine) transporter which allows maximal improvement of psychiatric symptoms while reducing adverse dopaminergic or noradrenergic effects including side effects by 1%, 3%, 10%, 20%, 30%, 50% or greater, up to a 75%, 80%, 90%, or 95% or greater over use of unbalanced triple reuptake inhibitors with higher rates of inhibition for dopamine or noradrenaline reuptake inhibitors.

The use of (+)-1-(3,4-dichlorophenyl)-3-azabicyclo[3.1.0]hexane will result in reuptake inhibition of the 5-HT transporter in individuals of about 10%, 15%, 20%, 30%, 50% or greater, up to a 75%, 80%, 90%, or 95% or greater than reuptake inhibition of the NE transporter or the DA transporter. In some embodiments reuptake inhibition of the 5HT transporter will be more than about 100% greater than reuptake inhibition of the DA or NE transporter in a particular individual. In some embodiments, reuptake inhibition of the 5-HT transporter will be two, three, four, five, six, seven or eight fold greater than the reuptake inhibition of the DA transporter. In other embodiments, reuptake inhibition of the 5-HT transporter will be one and half or twice that of the NE transporter. Reuptake inhibition of the NE transporter may be about 10%, 15%, 20%, 30%, 50% or greater, up to a 75%, 80%, 90%, or 95% or greater than reuptake inhibition of the DA transporter. In some embodiments, reuptake inhibition of the NE transporter may be two, three or four times greater than the reuptake inhibition of the DA transporter.

The use of (+)-1-(3,4-dichlorophenyl)-3-azabicyclo[3.1.0]hexane will result in binding of the 5-HT transporter in individuals at levels of about 10%, 15%, 20%, 30%, 50% or greater, up to a 75%, 80%, 90%, or 95% or greater than binding of the NE transporter or the DA transporter. In some embodiments, binding of the 5-HT transporter will be more than about 100% greater than the binding of the NE transporter or the DA transporter. In some embodiments, binding of the 5-HT transporter will be two, three, four, five, six, seven or eight fold greater than the binding of the DA transporter. In other embodiments, binding of the 5-HT transporter will be one and half or twice that of the NE transporter. Binding of the NE transporter may be about 10%, 15%, 20%, 30%, 50% or greater, up to a 75%, 80%, 90%, or 95% or greater than binding of the DA transporter in treated individuals. In some embodiments, binding of the NE transporter may be two, three or four times greater than binding of the DA transporter in an individual. The relative binding as determined by K, of 5-HT may be slightly higher, substantially higher, or significantly higher than the binding of the DA transporter or NE transporter alone or in combination.

(+)-1-(3,4-dichlorophenyl)-3-azabicyclo[3.1.0]hexane and pharmaceutically acceptable active salts, polymorphs, glycosylated derivatives, metabolites, solvates, hydrates, and/or prodrugs of (+)-1-(3,4-dichlorophenyl)-3-azabicyclo[3.1.0]hexane are useful for treating or preventing endogenous disorders alleviated by inhibiting dopamine and/or norepinephrine and/or serotonin reuptake. Such disorders include, but are not limited to, attention-deficit disorder, depression, anxiety disorder, panic disorder, post-traumatic stress disorder, obsessive compulsive disorder, schizophrenia and allied disorders, anxiety, obesity, tic disorders, Parkinson's disease, tic disorders, Parkinson's disease, chronic pain, attention deficit hyperactivity disorder (ADHD) and addictive and substance abuse disorders.

Disorders alleviated by inhibiting dopamine and/or norepinephrine and/or serotonin reuptake are not limited to the specific disorders described herein., and the compositions and methods of the invention will be understood or readily ascertained to provide effective treatment agents for treating and/or preventing a wide range of additional disorders and associated symptoms. For example, the compounds of the invention will provide promising candidates for treatment and/or prevention of depression, attention deficit hyperactivity disorder and related symptoms, as well as forms and symptoms of alcohol abuse, drug abuse, cognitive disorders, obsessive compulsive behaviors, learning disorders, reading problems, gambling addiction, manic symptoms, phobias, panic attacks, oppositional defiant behavior, conduct disorder, academic problems in school, smoking, abnormal sexual behaviors, schizoid behaviors, somatization, depression, sleep disorders, general anxiety, stuttering, and tic disorders (See, for example, U.S. Pat. No. 6,132,724). Additional disorders contemplated for treatment employing the compositions and methods of the invention are described, for example, in the Quick Reference to the Diagnostic Criteria From DSM-IV ((Diagnostic and Statistical Manual of Mental Disorders, Fourth Edition), The American Psychiatric Association, Washington, D.C., 2000, 358 pages.) Cognitive disorders for treatment and/or prevention according to the invention, include, but are not limited to, Attention-Deficit/Hyperactivity Disorder, Predominately inattentive Type; Attention-Deficit/Hyperactivity Disorder, Predominately Hyperactivity-Impulsive Type; Attention-Deficit/Hyperactivity Disorder, Combined Type; Attention-Delicit/Hyperactivity Disorder not otherwise specified (NOS); Conduct Disorder; Oppositional Defiant Disorder; and Disruptive Behavior Disorder not otherwise specified (NOS). Depressive disorders amenable for treatment and/or prevention according to the invention include, but are not limited to, Major Depressive Disorder, Recurrent; Dysthymic Disorder; Depressive Disorder not otherwise specified (NOS); and Major Depressive Disorder, Single Episode. Addictive disorders amenable for treatment and/or prevention employing the methods and compositions of the invention include, but are not limited to, eating disorders, impulse control disorders, alcohol-related disorders, nicotine-related disorders, amphetamine-related disorders, cannabis-related disorders, cocaine-related disorders, hallucinogen use disorders, inhalant-related disorders, and opioid-related disorders, all of which are further sub-classified as listed below. Substance abuse disorders include, but are not limited to alcohol-related disorders, nicotine-related disorders, Amphetamine-related disorders, cannabis-related disorders, cocaine-related disorders, hallucinogen-use disorders, inhalant-related disorders, and opioid-related disorders.

By virtue of their multiple reuptake inhibitor activity, the novel compounds of the present invention are thus useful in a wide range of veterinary and human medical applications, in particular for treating and/or preventing a wide array of disorders and/or associated symptom(s) alleviated by inhibiting dopamine and/or norepinephrine and/or serotonin reuptake. The unbalanced serotonin-norepinephrine-dopamine reuptake inhibition ratio of ˜1:2:8, respectively of (+)-1-(3,4-dichlorophenyl)-3-azabicyclo[3.1.0]hexane (Skolnick et al., 2003) provides several advantages in comparison to a balanced triple reuptake inhibitor and allows for higher dosages of (+)-1-(3,4-dichlorophenyl)-3-azabicyclo[3.1.0]hexane to be used without triggering the dopaminergic or norepinephrine side effects such as elevated heart rate, increased blood pressure, nausea, vomiting, insomnia and hypomania seen in similar dosages of balanced triple reuptake inhibitors.

Furthermore, the compositions of the present invention are effective in the treatment of those who have been previously treated for disorders affected by monoamine neurotransmitters such as depression. The compositions are additionally effective in the treatment of those who have had refractory experiences with prior treatments, i.e. individuals who have not responded, responded insufficiently, been unable to tolerate previous treatment(s) or who have otherwise responded in an unsatisfactory manner to other medications affecting monoamine neurotransmitters such as anti-depressants including, but not limited to, tri-cyclic antidepressants (TCAs), specific monoamine reuptake inhibitors, selective serotonin reuptake inhibitors, selective norepinephrine or noradrenaline reuptake inhibitors, selective dopamine reuptake inhibitors, serotonin-norepinephrine reuptake inhibitors, norepinephrine-dopamine reuptake inhibitors, multiple monoamine reuptake inhibitors, monoamine oxidase inhibitors, atypical antidepressants, atypical antipsychotics, anticonvulsants, or opiate agonists. Individuals may have been refractory to previous treatment(s) for any reason. In some embodiments, refractory individuals may have failed to respond or failed to respond sufficiently to a previous treatment. In one embodiment, a refractory individual may have treatment resistant depression. In other embodiments, a refractory individual may have responded to the initial treatment, but not succeed in entering remission from the treatment. In some embodiments, refractory individuals may have been unable to continue taking the medication due to intolerance of the medication including side effects such as, but not limited to, sexual dysfunction, weight gain, insomnia, dry mouth, constipation, nausea and vomiting, dizziness, memory loss, agitation, anxiety, sedation, headache, urinary retention, or abdominal pain.

Within additional aspects of the invention, combinatorial formulations and coordinate administration methods are provided which employ an effective amount of a (+)-1-(3,4-dichlorophenyl)-3-azabicyclo[3.1.0]hexane (or a pharmaceutically effective salt, solvate, hydrate, polymorph, or prodrug thereof), and one or more additional active agent(s) that is/are combinatorially formulated or coordinately administered with the compound of the invention—yielding a combinatorial formulation or coordinate administration method that is effective to modulate, alleviate, treat or prevent a targeted disorder, or one or more symptom(s) thereof, in a mammalian subject. Exemplary combinatorial formulations and coordinate treatment methods in this context comprise a therapeutic compound of the invention in combination with one or more additional or adjunctive treatment agents or methods for treating the targeted disorder or symptom(s), for example one or more antidepressant or anxiolytic agent(s) and/or therapeutic method(s).

In related embodiments of the invention., the compounds disclosed herein can be used in combination therapy with at least one other therapeutic agent or method. In this context, compounds of the invention can be administered concurrently or sequentially with administration of a second therapeutic agent, for example a second agent that acts to treat or prevent the same, or different, disorder or symptom(s) for which the compound of the invention is administered. The compound of the invention and the second therapeutic and/or psychotherapeutic agent can be combined in a single composition or administered in different compositions. The second therapeutic and/or psychotherapeutic agent may also be effective for treating and/or preventing a disorder or associated symptom(s) by inhibiting dopamine and/or norepinephrine and/or serotonin reuptake. The coordinate administration may be done simultaneously or sequentially in either order, and there may be a time period while only one or both (or all) active therapeutic agents, individually and/or collectively, exert their biological activities and therapeutic effects. A distinguishing aspect of all such coordinate treatment methods is that the compound of the invention exerts at least some detectable therapeutic activity toward alleviating or preventing the targeted disorder or symptom(s), as described herein, and/or elicit a favorable clinical response, which may or may not be in conjunction with a secondary clinical response provided by the secondary therapeutic agent. Often, the coordinate administration of a compound of the invention with a secondary therapeutic agent as contemplated herein will yield an enhanced therapeutic response beyond the therapeutic response elicited by either or both the compound of the invention and/or secondary therapeutic agent alone.

In one embodiment, combination therapy involves alternating between administering a compound of the present invention and a second therapeutic agent (i.e., alternating therapy regimens between the two drugs, e.g., at one week, one month, three month, six month, or one year intervals). Alternating drug regimens in this context will often reduce or even eliminate adverse side effects, such as toxicity, that may attend long-term administration of one or both drugs alone.

In certain embodiments of the invention, the additional psychotherapeutic agent is an antidepressant drug, which may include, for example, any species within the broad families of tri-cyclic antidepressants (TCAs) including, but not limited to, amitriptyline, imipramine, clomipramine, or desipramine; specific monoamine reuptake inhibitors; selective serotonin reuptake inhibitors (SSRIs) including, but not limited to, escitalopram, fluoxetine, fluvoxamine, sertraline, citalopram, vilazodone, and paroxetine; selective norepinephrine or noradrenaline reuptake inhibitors including but not limited to, tertiary amine tricyclics such as amitriptyline, clomipramine, doxepin, imipramine, (+)-trimipramine, and secondary amine tricyclics including amoxapine, atomoxetine, de sipramine, maprotiline, nortriptyline, and protriptyline; selective dopamine reuptake inhibitors; multiple monoamine reuptake inhibitors, e.g., that inhibit both serotonin and norepinephrine reuptake (SNRIs) including, but not limited to, venlafaxine, duloxetine, milnacipran, sibutramine, SEP-227162, LY 2216684, or inhibit both norepinephrine and dopamine, including but not limited to bupropion, amineptine, prolintane, dexmethylphenidate or pipradrol or those that inhibit both serotonin and dopamine; monoamine oxidase inhibitors (MAOIs); and indeterminate (atypical) antidepressants. The additional psychotherapeutic agent may additionally include atypical antipsychotics including, but not limited to, aripiprazole, ziprasidone, risperidone, quetiepine, or olanzapine or anticonvulsants including but not limited to gabopentin, pregabalin, lamotrigine, carbamazepine, oxcarbazepine, valproate, levetriacetam, and topiramate. Additional psychotherapeutic agents may additionally include opiate agonists including, but not limited to, buprenorphine, methadone and LAAM. Exemplary anxiolytics include, but are not limited to, buspirone, benzodiazepines, selective serotonin reuptake inhibitors, azapirones, barbiturates, hydroxyzine, and pregabalin.

In other embodiments of combinatorial formulations and coordinate treatment methods provided herein, the secondary psychotherapeutic agent is an anti-attention-deficit-disorder treatment agent. Examples of useful anti-attention-deficit-disorder agents for use in these embodiments include, but are not limited to, methylphenidate; dextroamphetamine and other amphetamines; tricyclic antidepressants, such as imipramine, desipramine, and nortriptyline; and psychostimulants, such as pemoline and deanol.

In additional embodiments of combinatorial formulations and coordinate treatment methods provided herein, the secondary psychotherapeutic agent is an anti-addictive-disorder or anti-substance abuse agent. Examples of useful anti-addictive-disorder agents include, but are not limited to, tricyclic antidepressants; glutamate antagonists, such as ketamine HCl, dextromethorphan, dextrorphan tartrate and dizocilpine (MK801); degrading enzymes, such as anesthetics and aspartate antagonists; GABA agonists, such as baclofen and muscimol HBr; reuptake blockers; degrading enzyme blockers; glutamate agonists, such as D-cycloserine, carboxyphenylglycine, L-glutarnic acid, and cis-piperidine-2,3-dicarboxylic acid; aspartate agonists; GABA antagonists such as gabazine (SR-95531), saclofen, bicuculline, picrotoxin, and (+) apomorphine HCl; and dopamine antagonists, such as spiperone HCl, haloperidol, and (−) sulpiride; anti-alcohol agents including, but not limited to, disulfuram and naltrexone; anti-nicotine agents including but not limited to, clonidine; anti-opiate agents including, but not limited to, methadone, clonidine, lofexidine, levomethadyl acetate HCl, naltrexone, and buprenorphine; anti-cocaine agents including, but not limited to, desipramine, amantadine, fluoxidine, and buprenorphine; anti-lysergic acid diethylamide (“anti-LSD”) agent including but not limited to, diazepam; anti-1-(1-phenylcyclohexyl)piperidine (“anti-PCP”) agent including, but not limited to, haloperidol.

In other embodiments of combinatorial formulations and coordinate treatment methods provided herein, the secondary therapeutic agent is an appetite suppressant. Examples of useful appetite suppressants include, but are not limited to, fenfluramine, phenylpropanolamine, bupropion, and mazindol.

In yet additional embodiments of combinatorial formulations and coordinate treatment methods provided herein, the secondary therapeutic agent is an anti-Parkinson's-disease agent. Examples of useful anti-Parkinson's-disease agents include, but are not limited to dopamine precursors, such as levodopa, L-phenylalanine, and L-tyrosine; neuroprotective agents; dopamine agonists; dopamine reuptake inhibitors; anticholinergics such as amantadine and memantine; and 1,3,5-trisubstituted adamantanes, such as 1-amino-3,5-dimethyl-adamantane. (See, U.S. Pat. No. 4,122,193)

In further embodiments of combinatorial formulations and coordinate treatment methods provided herein, the secondary therapeutic agent is an anti-inflammatory agent. Examples of useful anti-inflammatory agents included, but are not limited to celecoxib, ibuprofen, ketoprofen, naproxen sodium, piroxicam, sulindac, aspirin, and nabumetone.

Suitable routes of administration for a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention include, but are not limited to, oral, buccal, nasal, aerosol, topical, transdermal, transdermal patch, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Suitable effective unit dosage amounts of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention for mammalian subjects may range from about 5 to about 1800 mg, about 10 to about 1800 mg, 25 to about 1800 mg, about 50 to about 1000 mg, about 75 to about 900 mg, about 100 to about 750 mg, or about 150 to about 500 mg. In certain embodiments, the effective dosage will be selected within narrower ranges of, for example, about 5 to about 10 mg, 10 to about 25 mg, about 30 to about 50 mg, about 10 to about 300 mg, about 25 to about 300 mg, about 75 to about 100 mg, about 100 to about 250 mg, or about 250 to about 500 mg. These and other effective unit dosage amounts may be administered in a single dose, or in the form of multiple daily, weekly or monthly doses, for example in a dosing regimen comprising from 1 to 5, or 2-3, doses administered per day, per week, or per month. In exemplary embodiments, dosages of about 10 to about 25 mg, about 30 to about 50 mg, about 25 to about 150, about 75 to about 100 mg, about 100 to about 250 mg, or about 250 to about 500 mg, are administered one, two, three, or four times per day. In more detailed embodiments, dosages of about 50-75 mg, about 100-200 mg, about 250-400 mg, or about 400-600 mg are administered once or twice daily. In alternate embodiments, dosages are calculated based on body weight, and may be administered, for example, in amounts from about 0.5 mg/kg to about 20 mg/kg per day, 1 mg/kg to about 15 mg/kg per day, 1 mg/kg to about 10 mg/kg per day, 2 mg/kg to about 20 mg/kg per day, 2 mg/kg to about 10 mg/kg per day or 3 mg/kg to about 15 mg/kg per day.

The amount, timing, and mode of delivery of compositions of the invention comprising an effective amount of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention will be routinely adjusted on an individual basis, depending on such factors as weight, age, gender, and condition of the individual, the acuteness of the condition to be treated and/or related symptoms, whether the administration is prophylactic or therapeutic, and on the basis of other factors known to effect drug delivery, absorption, pharmacokinetics, including half-life, and efficacy. An effective dose or multi-dose treatment regimen for the compounds of the invention will ordinarily be selected to approximate a minimal dosing regimen that is necessary and sufficient to substantially prevent or alleviate one or more symptom(s) of a neurological or psychiatric condition in the subject, as described herein. Thus, following administration of a (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention according to the formulations and methods herein, test subjects will exhibit a 10%, 20%, 30%, 50% or greater reduction, up to a 75-90%, or 95% or greater, reduction, in one or more symptoms associated with a targeted monoamine neurotransmitter influenced disorder or other neurological or psychiatric condition, compared to placebo-treated or other suitable control subjects.

Pharmaceutical dosage forms of a compound of the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Pharmaceutical dosage forms of (+)-1-(3,4-dichlorophenyl)-3-azabicyclo[3.1.0]hexane may include inorganic and organic acid addition salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; organic acid salts such as acetate, citrate, lactate, succinate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate, tartrate, gluconate and the like.

Within various combinatorial or coordinate treatment methods of the invention, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) may each be administered by any of a variety of delivery routes and modes, which may be the same or different for each agent.

An additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute episodes of mania.

The additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention can be prepared and administered in any of a variety of inhalation or nasal delivery forms known in the art. Devices capable of depositing aerosolized formulations of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of depression. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds of the invention into the blood stream directly after administering an effective amount of the compound to the nose, without requiring the product to be deposited in the lung. In addition, intranasal delivery can achieve direct, or enhanced, delivery of the active additional psychotherapeutic compound and/or (+)-1-(3,4-dichlorophenyl)-3-azabicyclo[3.1.0]hexane to the central nervous system. In these and other embodiments, intranasal administration of the compounds of the invention may be advantageous for treating disorders influenced by monoamine neurotransmitters, by providing for rapid absorption and delivery.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) of the present invention. Topical compositions may comprise a compound of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound of the present invention dissolved or dispersed in water or other solvent or liquid to be incorporated in the topical composition or delivery device. It can be readily appreciated that the transdermal route of administration, such as by a transdermal patch, may be enhanced by the use of a dermal penetration enhancer known to those skilled in the art. Formulations suitable for such dosage forms incorporate excipients commonly utilized therein, particularly means, e.g. structure or matrix, for sustaining the absorption of the drug over an extended period of time, for example 24 hours.

Yet additional formulations of a compound of the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

Within exemplary compositions and dosage forms of the invention, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) for treating disorders disclosed herein is/are administered in an extended release or sustained release formulation. In these formulations, the sustained release composition of the formulation provides therapeutically effective plasma levels of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) over a sustained delivery period of approximately 8 hours or longer, or over a sustained delivery period of approximately 18 hours or longer, up to a sustained delivery period of approximately 24 hours or longer.

In exemplary embodiments, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is/are combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises an oral sustained release dosage composition for administering an additional psychotherapeutic compound and/or (+)-1-(3,4-dichlorophenyl)-3-azabicyclo[3.1.0]hexane agent (including (+)-1-(3,4-dichlorophenyl)-3-azabicyclo[3.1.0]hexane and pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) according to the invention. In a related aspect, the invention comprises a method of reducing one or more side effects that attend administration of an oral dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) compound by employing a sustained release formulation. Within these methods, an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is provided in a sustained release oral dosage form and the dosage form is introduced into a gastrointestinal tract of a mammalian subject presenting with a disorder amenable to treatment using the subject therapeutic drug, by having the subject swallow the dosage form. The method further includes releasing the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in a sustained, delayed, gradual or modified release delivery mode into the gastrointestinal tract (e.g., the intestinal lumen) of the subject over a period of hours, during which the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) reach(es), and is/are sustained at, therapeutic concentration(s) in a blood plasma, tissue, organ or other target site of activity (e.g., a central nervous system tissue, fluid or compartment) in the patient. When following this method, the side effect profile of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is less than a side effect profile of an equivalent dose of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) administered in an immediate release oral dosage form.

In certain embodiments, the additional psychotherapeutic compound and/or (+)-1-(3,4-dichlorophenyl)-3-azabicyclo[3.1.0]hexane is/are released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) following administration to a mammalian subject in a desired dosage amount (e.g., 5, 10, 25, 50, 100 200, 400, 600, or 800 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) following administration to a mammalian subject in a desired dosage amount (e.g., 5, 10, 25, 50, 100, 200, 400, 600, or 800 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, the active additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is/are released from the compositions and dosage forms of the invention and delivered into the blood plasma or other target site of activity in the subject (including, but not limited to, areas of the brain such as the thalamus, striatum, ventral tegmental area, cortical areas, hippocampus, hypothalamus, or nucleus accumbens) in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of an additional psychotherapeutic compound and/or (+)-1-(3,4-dichlorophenyl)-3-azabicyclo[3.1.0]hexane agents are provided, wherein the compositions and dosage forms, after ingestion, provide a curve of concentration of the additional psychotherapeutic compound and/or (+)-1-(3,4-dichlorophenyl)-3-azabicyclo[3.1.0]hexane agents over time, the curve having an area under the curve (AUC) which is approximately proportional to the dose of the additional psychotherapeutic compound and/or (+)-1-(3,4-dichlorophenyl)-3-azabicyclo[3.1.0]hexane agents administered, and a maximum concentration (Cₘₐₓ) that is proportional to the dose of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) administered.

In other detailed embodiments, the Cₘₐₓ of the active additional psychotherapeutic compound and/or (+)-1-(3,4-dichlorophenyl)-3-azabicyclo[3.1.0]hexane agents provided after oral delivery of a composition or dosage form of the invention is less than about 80%, often less than about 75%, in some embodiments less than about 60%, or 50%, of a Cₘₐₓ obtained after administering an equivalent dose of the active compound in an immediate release oral dosage form.

Within exemplary embodiments of the invention, the compositions and dosage forms containing the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) and a sustained release vehicle, matrix, binder, or coating will yield sustained delivery of the active compound such that, following administration of the composition or dosage form to a mammalian treatment subject, the Cₘₐₓ of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in the treatment subject is less than about 80% of a Cₘₐₓ provided in a control subject after administration of the same amount of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in an immediate release formulation.

As used herein, the term “immediate release dosage form” refers to a dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) wherein the active compound readily dissolves upon contact with a liquid physiological medium, for example phosphate buffered saline (PBS) or natural or artificial gastric fluid. In certain embodiments, an immediate release formulation will be characterized in that at least 70% of the active compound will be dissolved within a half hour after the dosage form is contacted with a liquid physiological medium. In alternate embodiments, at least 80%, 85%, 90% or more, or up to 100%, of the active compound in an immediate release dosage form will dissolve within a half hour following contact of the dosage form with a liquid physiological medium in an art-accepted in vitro dissolution assay. These general characteristics of an immediate release dosage form will often relate to powdered or granulated compositions of an additional psychotherapeutic compound and/or (+)-1-(3,4-dichlorophenyl)-3-azabicyclo[3.1.0]hexane agents in a capsulated dosage form, for example in a gelatin-encapsulated dosage form, where dissolution will often be relatively immediate after dissolution/failure of the gelatin capsule. In alternate embodiments, the immediate release dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an immediate release dosage form will include compositions wherein the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is not admixed, bound, coated or otherwise associated with a formulation component that substantially impedes in vitro or in vivo dissolution and/or in vivo bioavailability of the active compound. Within certain embodiments, the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of an additional psychotherapeutic compound and/or (+)-1-(3,4-dichlorophenyl)-3-azabicyclo[3.1.0]hexane agents in a formulation containing such materials by at least 5%, often at least 10%, and up to at least 15%-20% compared to dissolution of the additional psychotherapeutic compound and/or (+)-1-(3,4-dichlorophenyl)-3-azabicyclo[3.1.0]hexane agents when provided in a composition that is essentially free of such materials.

In alternate embodiments of the invention, an immediate release dosage form of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation.” In exemplary embodiments, an immediate release formulation of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) formulation according to this aspect of the invention will exhibit rapid dissolution characteristics according to BCS Guidance parameters, such that at least approximately 85% of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in the formulation will go into a test solution within about 30 minutes at pH 1, pH 4.5, and pH 6.8.

The compositions, dosage forms and methods of the invention thus include novel tools for coordinate treatment of disorders involving monoamine neurotransmitters by providing for sustained release and/or sustained delivery of the additional psychotherapeutic agent and/or (+)-1-(3,4-dichlorophenyl)-3-azabicyclo[3.1.0]hexane agents. As used herein, “sustained release” and “sustained delivery” are evinced by a sustained, delayed, extended, or modified, in vitro or in vivo dissolution rate, ins vivo release and/or delivery rate, and/or in vivo pharmacokinetic value(s) or profile.

The sustained release dosage forms of the present invention can take any form as long as one or more of the dissolution, release, delivery and/or pharmacokinetic property(ies) identified above are satisfied. Within illustrative embodiments, the composition or dosage form can comprise an additional psychotherapeutic compound and/or (+)-1-(3,4-dichlorophenyl)-3-azabicyclo[3.1.0]hexane agents combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus provides a broad range of sustained release compositions and dosage forms comprising an additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane), which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with the additional psychotherapeutic compound and/or (+)-1-(3,4-dichlorophenyl)-3-azabicyclo[3.1.0]hexane agents to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

In a particular embodiment described below in Example XII, a formulation is provided for an oral unit dosage extended release tablet of an HCl salt of (+)-1-(3,4-dichlorophenyl)-3-azabicyclo[3.1.0]hexane. In that formulation hydroxypropylmethyl cellulose is used as a sustained release vehicle, while microcrystalline cellulose and starch is used as a carrier. In particular, that formulation of a 350 mg tablet contains 100 mg of (+)-1-(3,4-dichlorophenyl)-3-azabicyclo[3.1.0]hexane (HCl salt), 105 mg of Methocel Premium CR K4 or K100, 71.5 mg Microcrystalline Cellulose, 70 mg pregelatinized starch 1500, 1.75 mg colloidal silicon dioxide, 1.75 mg magnesium stearate, and an optional coating, such as Opadry II White. Thus, that formulation uses 30% hydroxypropylmethyl cellulose (% of total weight of the tablet ingredients). Accordingly, an oral extended release tablet of (+)-1-(3,4-dichlorophenyl)-3-azabicyclo[3.1.0]hexane HCl or other pharmaceutically acceptable salt will include an amount of about 15-45%, 25-35%, or 30% of hydroxypropyl methyl cellulose of total weight of the tablet ingredients. An oral extended release tablet of (+)-1-(3,4-dichlorophenyl)-3-azabicyclo[3.1.0]hexane HCl or other pharmaceutically acceptable salt will further contain about 25 to 200 mg, 50 to 150 mg, or 100 mg of an active ingredient of (+)-1-(3,4-dichlorophenyl)-3-azabicyclo[3.1.0]hexane HCl or other pharmaceutically acceptable salt. An oral extended release tablet of (+)-1-(3,4-dichlorophenyl)-3-azabicyclo[3.1.0]hexane HCl or other pharmaceutically acceptable salt will additionally contain from about 30-50% or 40% of pharmaceutically acceptable carrier. An extended release profile of the formulation of Example XII is demonstrated by dissolution studies shown in Example XIII. Those studies demonstrate that the formulation of Example XII does indeed achieve an extended release commensurate with a tablet to be administered once per day.

Additional polymeric materials for use as sustained release vehicles, matrices, binders, or coatings within the compositions and dosage forms of the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from copolymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-coglycolic acid) (D PLGA) and poly(L-lactic acid-co-glycolic acid) (L PLGA). Other biodegradable or bioerodable polymers for use within the invention include such polymers as poly(ε-caprolactone), poly(ε-caprolactone-CO-lactic acid), poly(ε-caprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly(ester ureas), poly(2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms comprise an additional psychotherapeutic compound and/or (+)-1-(3,4-dichlorophenyl)-3-azabicyclo[3.1.0]hexane agents coated on a polymer substrate. The polymer can be an erodible or a nonerodible polymer. The coated substrate may be folded onto itself to provide a bilayer polymer drug dosage form. For example the additional psychotherapeutic compound and/or (+)-1-(3,4-dichlorophenyl)-3-azabicyclo[3.1.0]hexane agents can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) loaded into a polymer that releases the drug(s) by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, the compositions and dosage forms comprise an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising an additional psychotherapeutic compound and/or (+)-1-(3,4-dichlorophenyl)-3-azabicyclo[3.1.0]hexane agents and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14^{th} ed, pp 1626-1628 (1970); Fincher, J. Pharm. Sci. 1968, 57, 1825-1835; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In another embodiment of the invention, the dosage form comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) agent, e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising an additional psychotherapeutic agent and/or (+)-1-(3,4-dichlorophenyl)-3-azabicyclo[3.1.0]hexane agents. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing sustained release compositions and dosage forms comprising an additional psychotherapeutic agent and/or (+)-1-(3,4-dichlorophenyl)-3-azabicyclo[3.1.0]hexane agents. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al., U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for sustained release delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, the dosage form comprises an osmotic dosage form, which comprises a semi-permeable wall that surrounds a therapeutic composition comprising the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane). In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semipermeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) composition layer from the dosage form, and at least one passageway in the wall for releasing the drug composition. This osmotic system delivers the active compound by imbibing fluid through the semipermeable wall at a fluid imbibing rate determined by the permeability of the semipermeable wall and the osmotic pressure across the semipermeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semipermeable wall comprises a composition that is permeable to the passage of fluid and impermeable to passage of the additional psychotherapeutic agent and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane). The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) from the dosage form to the patient at a zero order rate of release over a period of up to about 24 hours. As used herein, the expression “passageway” comprises means and methods suitable for the metered release of an additional psychotherapeutic compound and/or (+)-1-(3,4-dichlorophenyl)-3-azabicyclo[3.1.0]hexane agents from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference).

Within other aspects of the invention, microparticle, microcapsule, and/or microsphere drug delivery technologies can be employed to provide sustained release delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) within the compositions, dosage forms and methods of the invention. A variety of methods is known by which an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). These documents disclose methods that can be readily implemented to prepare microparticles containing an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc such as Polyvidon K30 (trade mark), magnesium stearate, and silicon dioxide.

Additional enteric coating materials for mediating sustained release of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention including coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethylcellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxlated ethylene-vinylacetate, cellulose acetate butyrate, polyallcenes, polyethers, polysulfones, polyethersulfones, polystyrenes, polyvinyl halides, polyvinyl esters and ethers, natural waxes and synthetic waxes.

In additional embodiments of the invention, sustained release of the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are described, for example, in International Publications WO 95/20946; WO 94/06416; and WO 98/05305 (each incorporated herein by reference). Other multi-component dosage forms for providing sustained delivery of an additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) include tablet formulations having a core containing the active compound coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising the additional psychotherapeutic compound and/or (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™, Methocel E4M™, Methocel K15M™ and Methocel K100M™. Other known release-retarding polymers which may be incorporated within this and other embodiments of the invention to provide a sustained release composition or dosage form of an additional psychotherapeutic compound and/or (+)-1-(3,4-dichlorophenyl)-3-azabicyclo[3.1.0]hexane agents include, hydrocolloids such as natural or synthetic gums, cellulose derivatives other than those listed above, carbohydrate-based substances such as acacia, gum tragacanth, locust bean gum, guar gum, agar, pectin, carrageenan, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference). In other embodiments, an oral sustained release pump may be used (see, e.g., Langer, supra; Sefton, 1987, CRC Crit. Ref Biomed. Eng. 14:201; and Saudek et al., 1989, N. Engl. J. Med. 321:574, each incorporated herein by reference).

The pharmaceutical compositions and dosage forms of the current invention will typically be provided for administration in a sterile or readily sterilizable, biologically inert, and easily administered form.

In other embodiments the invention provides pharmaceutical kits for reducing symptoms in a human subject suffering from a disorder affected by monoamine neurotransmitters, including depression. The kits comprise the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) therapeutic agent in an effective amount, and a container means for containing the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) for coordinate administration to the said subject (for example a container, divided bottle, or divided foil pack). The container means can include a package bearing a label or insert that provides instructions for multiple uses of the kit contents to treat the disorder and reduce symptoms in the subject. In more detailed embodiments, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are admixed or co-formulated in a single, combined dosage form, for example a liquid or solid oral dosage form. In alternate embodiments, the additional psychotherapeutic agent and (+)-1-(3,4-dichlorophenyl)-3-azabicyclo[3.1.0]hexane agent (including pharmaceutically acceptable active salts polymorphs, glycosylated derivatives, metabolites, solvates, hydrates, and/or prodrugs of (+)-1-(3,4-dichlorophenyl)-3-azabicyclo[3.1.0]hexane) are contained in the kit in separate dosage forms for coordinate administration. An example of such a kit is a so-called blister pack. Blister packs are well-known in the packaging industry and are widely used for the packaging of pharmaceutical dosage forms (tablets, capsules and the like).

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The following examples illustrate certain aspects of the invention, but are not intended to limit in any manner the scope of the invention.

### Example 1

### Preparation of 1(3,4-dichlorophenyl)-3-azabicyclo[3.1.0]hexane

As described in U.S. Pat. No. 4,231,935, a solution of 59.5 g of 3,4-dichlorophenylacetic acid in 500 ml of absolute ethanol is saturated with anhydrous hydrogen chloride and then heated at reflux for 2 hours. The mixture is concentrated under reduced pressure to 200 ml, diluted with 200 ml of water and neutralized with concentrated ammonium hydroxide. This aqueous mixture is extracted 3 times with chloroform. Concentration and decolorization of the chloroform extracts gives ethyl 3,4-dichlorophenylacetate as a yellow oil.

In a three-necked flask fitted with a Nichrome stirrer and a reflux condenser is placed 7.0 g of ethyl 3,4-dichlorophenylacetate, 5.9 g of N-bromosuccinimide, 0.1 g of benzoyl peroxide and 150 ml of carbon tetrachloride. The reaction mixture is heated at reflux for 18 hours, cooled and filtered. The carbon tetrachloride filtrate is concentrated under reduced pressure to give a deep orange liquid. Vacuum distillation at 115°-120° C. (0.5 mm) gives ethyl α-bromo-3,4-dichlorophenylacetate as a pale yellow liquid.

This product is converted to diethyl cis-1-(3,4-dichlorophenyl)-1,2-cyclopropanedicarboxylate by the method of L. L. McCoy, J.A.C.S., 80, 6568 (1958).

A mixture of 150 g of this diester and 66 g of 85% KOH in 500 ml of water and 500 ml of ethanol is refluxed for 6 hours and then chilled in ice. The oily material is extracted into ether and the aqueous layer is made acidic with 100 ml of 12 N hydrochloric acid. The oily lower layer crystallizes slowly to give a colorless crystalline cake. This is recrystallized from a mixture of ethanol and ethyl acetate to give colorless crystals of 1-(3,4-dichlorophenyl)-1,2-cyclopropanedicarboxylic acid.

A mixture of 30.3 g of this diacid and 12.6 g of urea in one liter of xylene is refluxed for 6 hours. The solvent is stripped under reduced pressure and the crystalline residue is slurried with water. The colorless crystals are collected by filtration, washed with water and air dried to give 1-(3,4-dichlorophenyl)-1,2-cyclopropanedicarboximide.

To 40 ml of 1 molar borane-tetrahydrofuran is added with stirring under nitrogen at 0° C. a solution of 2.56 g of this imide in 50 ml of tetrahydrofuran during 15 minutes. The solution is warmed in a steam bath for 1 hour and is then cooled in ice, and then 20 ml of 6 N hydrochloric acid is added, and the tetrahydrofuran is removed under reduced pressure. The residue is made basic with 75 ml of 5 N sodium hydroxide and this is extracted with ether. The extract is dried over magnesium sulfate, filtered, and the filtrate is saturated with hydrogen chloride. The precipitated crystals are collected by filtration and are recrystallized from isopropyl alcohol to give 1.70 g of 1-(3,4-dichlorophenyl)-3-azabicyclo[3.1.0]hexane hydrochloride as colorless crystals, m.p. 180°-181° C.

### Example II

### (+) 1(3,4-dichlorophenyl)-3-azabicyclo[3.1.0]hexane

To 279 mg of (±)-1-(3,4-dichlorophenyl)-3-azabicyclo[3.1.0]hexane hydrochloride obtained using the methods described above or in Epstein et al., J. Med. Chem., 24:481-490 (1981) was added 7 mL of 9:1 hexane:isopropyl alcohol, followed by 8 drops of diethylamine. To the resulting mixture was added isopropyl alcohol, dropwise, until a solution was obtained. The solution was concentrated to a volume of 6 mL using a stream of helium gas, and six 1-mL portions of the concentrate were subjected to high-performance liquid chromatography using an HPLC instrument equipped with a 1 cm×25 cm Daicel CHIRALPAK AD column (Chiral Technologies, Inc., Exton, Pa.). Elution was carried out at ambient temperature using 95:5 (v/v) hexane:isopropyl alcohol solution containing 0.05% diethylamine as a mobile phase at a flow rate of 6 mL/min. The fraction eluting at about 21.5 to 26 minutes was collected and concentrated to provide a first residue, which was dissolved in a minimal amount of ethyl acetate. Using a stream of nitrogen, the ethyl acetate solution was evaporated to provide a second residue, which was dissolved in 1 mL of diethyl ether. To the diethyl ether solution was added 1 mL diethyl ether saturated with gaseous hydrochloric acid. A colorless precipitate formed, which was filtered, washed with 2 mL of diethyl ether and dried to provide 73.4 mg of (+)-1-(3,4-dichlorophenyl)-3-azabicyclo[3.1.0]hexane hydrochloride: optical rotation [α]²⁵_{D}=+ 60° in methanol at 2 mg/mL; 99.7% enantiomeric excess. (See, U.S. Pat. No. 6,372,919)

### Example III

### Preparation of (1R,5S)-(+)-1-(3,4-dichlorophenyl)-3-azabicyclo[3,10]hexane

To a solution of 3,4-dichlorophenylacetonitrile (3.50 kg) and S-(+)-epichlorohydrin (2.22 kg) in THF (18.5 L) at −15° C. under atmosphere of N₂ was added NaHMDS (16.5 L, 2M in THF) dropwise over 3 h. The reaction mixture was stirred for 3 h at −15° C., then, overnight at −5° C. BH₃-Me_{2S} (neat, 10M, 4.4 L) was added over 2 h. The reaction mixture was then gradually warmed to 40° C. over 3 h. After aging 1.5 h at 40° C., the reaction mixture was cooled to 20-25° C. and slowly quenched into a 2N HCl solution (27.7 L). The quenched mixture was then aged for 1 h at 40° C. Concentrated NH₄OH (6.3 L) was added and the aqueous layer was discarded. i-PrOAc (18.5 L) and 5% dibasic sodium phosphate (18.5 L) were charged. The organic phase was then washed with saturated brine (18.5 L), azeotropically dried and solvent-switched to i-PrOAc (ca. 24.5 L) in vacuum.

The above crude amino alcohol solution in i-PrOAc was slowly subsurface-added to a solution of SOCl₂ (22.1 mol, 1.61 L) in i-PrOAc (17.5 L) at ambient temperature over 2 h. After aging additional 1-5 h, 5.0 N NaOH (16.4 L) was added over 1 h while the batch temperature was maintained at <30° C. with external cooling. The two-phase reaction mixture was stirred for 1 h at ambient temperature to allow pH to stabilize (usually to 8.5-9.0) with NaOH pH titration. The organic phase was washed with 40% aqueous i-PrOH (21 L) followed by water (10.5 L). Conc. HCl (1.69 L) was added. The aqueous i-PrOAc was azeotropically concentrated in vacuum to ca. 24.5 L. Methylcyclohexane (17.5 L) was added dropwise over 2 h. The wet cake was displacement-washed with 7 L of 40% methylcyclohexane/1-PrOAc followed by a slurry wash (7 L, i-PrOAc) and a displacement wash (7 L, i-PrOAc). Typical isolated yield: 57-60% corrected with wt %: 87-99.5% (based on HCl salt).

(1R,5S)-(+)-1-(3,4-dichlorophenyl)-3-azabicyclo[3,10]hexane HCl salt (5.0 kg) was dissolved in i-PrOH (14.25 L) and water (0.75 L) at 55° C. Seeds (50 g) were added at 48-50° C. The batch was allowed to cool to ambient temperature (20° C.) over 2-4 h. MeOBu-t (37 L) was added dropwise over 2 h. After aging 1 h at 20° C., the batch was filtered. The wet cake was displacement-washed with 10 L of 30% i-PrOH in MeOBu-t followed by 2×7.5 L 10% i-PrOH in MeOBu-t (slurry wash, then displacement wash). The wet cake was suction dried under N₂ (10-50 RH %) at ambient temperature to give the hemihydrate HCl salt of (1R,5S)-(+)-1-(3,4-dichlorophenyl)-3-azabicyclo[3,10]hexane. Typical yield: 92%. ¹H-NMR (400 MHz, d₄-MeOH): Δ 7.52 (d, J=2.2 Hz, 1H), 7.49 (d, J=8.4 Hz, 1H), 7.26 (dd, J=2.1, 8.4 Hz, 1H), 3.78 (d, J=11.4 Hz, 1H), 3.69 (dd, J=3.9, 11.3 Hz, 1H), 3.62 (dd, J=1.4, 11.3 Hz, 1H), 3.53 (d, J=11.4 Hz, 1H), 2.21 (m, 1H), 1.29 (t, J=7.5 Hz, 1H), 1.23 (dd, J=4.9, 6.5 Hz, 1H). ¹³C-NMR (100 MHz, d₄-MeOH): Δ 141.0, 133.7, 132.2, 132.0, 130.6, 128.4, 51.7, 49.1, 31.8, 24.9, 16.5. Anal. Calcd for C₁₁H₁₃Cl₃NO_{0.5}: C, 48.29; H, 4.79; N, 5.12; Cl, 38.88. Found: C, 48.35; H, 4.87; N, 5.07; 38.55. (See U.S. patent application Ser. No. 11/740,667)

### Example IV

### Method of Manufacture of (+)-1-(3,4-dichlorophenyl)-3-azabicyclo[3.1.0]hexane hydrochloride

(+)-1-(3,4-dichlorophenyl)-3-azabicyclo[3.1.0]hexane hydrochloride may also be manufactured according to the procedure described in U.S. patent application Ser. No. 12/428,399 as follows:

### Step 1: Synthesis of α-bromo-3,4-dichlorophenylacetic acid methyl ester

100 kg 3,4-dichlorophenylacetonitrile was added in portions over 1.25 hours to a mixture of 12 kg water and 140 kg 98% sulfuric acid. Exotherm was allowed to 65° C. maximum, and the reaction mix was maintained at 60-65° C. for 30 minutes. After cooling to 50° C., 80 kg methanol was slowly added over 25-30 minutes. The mixture was warmed to 92-98° C., and maintained at this temperature for an additional three hours. After cooling to 35° C., the reaction mixture was quenched into an agitated mixture (precooled to 0-5° C.) of 150 L ethylene dichloride and 250 L water. The reactor and lines were washed with water into the quench mix, which was agitated 5 minutes and allowed to stratify. The lower organic phase was separated, and the aqueous phase washed with 2×150 L ethylene dichloride. The combined organic phases were washed with 100 L water and then with aqueous sodium carbonate (3 kg sodium carbonate in 100 L water). The solution of crude ester was azeotropically “dried” in vacuo at 60-620 C, resulting in the collection of 100 L ethylene dichloride. A theoretical yield was assumed without isolation and the solution was used “as is” in the following bromination reaction.

A mixture of the solution (line-filtered) of crude methyl 3,4-dichlorophenylacetate (from above) and 88 kg 1,3-dibromo-1,3-dlmethylhydantoin (DBDMH) was warmed to 80° C., and a solution of 2.5 kg VAZO 52 in 15 L ethylene dichloride was added portion wise over a 5 hour period, maintaining 85-90° C. (under reflux). An additional 8.8 kg DBDMH was then added, and a solution of 0.5 kg VAZO 52 in 4 L ethylene dichloride was added portion wise over a 2.5 hour period, maintaining 85-90° C. (under reflux). Heating was then discontinued, and 350 L water was added with agitation. The mixture was allowed to stratify, the lower organic phase was separated and the aqueous phase was washed with 50 L ethylene dichloride. The combined organic phases were washed with aqueous thiosulfate (5.0 kg sodium thiosulfate in 150 L water), aqueous sodium carbonate (2.5 kg sodium carbonate in 150 L water), and dilute hydrochloric acid (5.4 L 32% HCl in 100 L water). The organic phase was line-filtered and distilled in vacuo to “dryness” (full vacuum to 83° C.). Residual ethylene dichloride was chased with 20 kg toluene (full vacuum at 83° C.). The crude α-bromo-3,4-dichlorophenylacetic acid methyl ester was taken up in 82 kg toluene, cooled to 40° C., and discharged to steel drums. The product was not isolated, and was used “as is” in Step 2. A theoretical yield was assumed for calculation purposes.

### Step 2: Synthesis of 1-(3,4-dichlorophenyl)-1,2-cyclopropane-dicarboxylic acid dimethyl ester

The crude α-bromo-3,4-dichlorophenylacetic acid methyl ester from Step 1 was mixed well with 55.6 kg methyl acrylate, and then the mixture was added to a precooled (−2° C.) mixture of 54.4 kg potassium methoxide in 500 L toluene (argon blanket) over 5.5 hours with good agitation and maintained at <10° C. After standing overnight (5 psig argon) with brine cooling (−5° C.), the cold reaction mixture was quenched into a mix of 250 L water and 30 kg 32% hydrochloric acid with good agitation. 200 L water and 2.5 kg potassium carbonate were added to the mixture with good agitation for an additional 30 minutes. After stratification, the lower aqueous phase was separated, and 150 L water and 1.0 kg potassium carbonate were added to the organic phase. The mixture was agitated 5 minutes and stratified. The lower aqueous phase was separated and discarded, as well as the interfacial emulsion, and the organic phase was washed with 100 L water containing 1 L 32% hydrochloric acid. After stratification and separation of the lower aqueous phase, the organic phase was line-filtered and distilled in vacuo to “dryness” (full vacuum at 65° C.). To the hot residue was added 70 kg methanol with agitation. The mix was cooled (seeding at +10° C.) to −5° C. and maintained at this temperature overnight. The cold thick suspension was suction-filtered (Nutsche), and the cake of 1-(3,4-dichlorophenyl)-1,2-cyclopropane-dicarboxylic acid dimethyl ester was suction dried, washed with 2×20 L hexane, suction dried for 30 minutes and air-dried on paper (racks) for 2 days at ambient conditions.

To the methanolic liquors was added 50 kg caustic soda flake portion wise over 8 hours with good agitation. After gassing and the slow exotherm (60° C. maximum) ceased, the heavy suspension was held at 50° C. for 1 hour. 100 L isopropanol was slowly added over 10 minutes, and then the mixture was agitated slowly overnight at ambient conditions. The solids were suction-filtered (Nutsche) and reslurried with 80 L methanol. The resulting 1-(3,4-dichlorophenyl)-1,2-cyclopropane-dicarboxylic acid disodium salt was suctioned-filtered (Nutsche), washed with methanol (40 L), suction dried for 1 hour and air-dried on paper (racks).

### Step 3: Synthesis of 1-(3,4-dichlorophenyl)-1,2-cyclopropane-dicarboxylic acid

A suspension of 42.0 kg 1-(3,4-dichlorophenyl)-1,2-cyclopropane-dicarboxylic acid disodium salt (from Step 2) and 120 L deionized water was warmed to 30-35° C., and the solution was line-filtered and neutralized with 30 kg 32% hydrochloric acid to precipitate the free dicarboxylic acid. 120 kg ethyl acetate was added, and the mix warmed to 40-50° C. to effect solution. The lower aqueous phase was separated and washed with 20 kg ethyl acetate. The combined organic extracts were washed with saturated sodium chloride (3 kg in 30 L water) and then distilled in vacuo to “dryness” (full vacuum to 70° C.). 60 kg ethylene dichloride was added to the warm residue, and the solution cooled with slow agitation at −5° C. overnight. Residual ethyl acetate was distilled (full vacuum to 43° C.) to yield a thick suspension, which was then cooled with full vacuum to −5° C. over a 2.5 hour period and then suction-filtered (Nutsche). The 1-(3,4-dichlorophenyl)-1,2-cyclopropane-dicarboxylic acid cake was washed with cold ethylene dichloride (2×5 L), followed by ambient ethylene dichloride (4×5 L). The dicarboxylic acid product was suction dried for 15 minutes and air-dried on paper (racks).

A mixture of 31.0 kg 1-(3,4-dichlorophenyl)-1,2-cyclopropane-dicarboxylic acid dimethyl ester (from Step 2), 40 L water, 35 kg methanol and 18.0 kg 50% caustic soda was warmed to 70-75° C. (under reflux) and maintained at 70-75° C. for 1.5 hours. 10 L water was then added, and the mixture was kept at 75-77° C. for an additional 2 hours. Methanol was slowly distilled off in vacuo to 70° C. to give a heavy suspension, which was then mixed with 80 L water to effect solution. The free dicarboxylic acid was precipitated with 31 kg of 32% hydrochloric acid and extracted with 100 kg ethyl acetate. The lower aqueous phase was separated and washed with 20 kg ethyl acetate. The combined organic phases were washed with 50 L water, and then saturated aqueous sodium chloride. Distillation in vacuo to 80° C. with full vacuum yielded a concentrate of 1-(3,4-dichlorophenyl)-1,2-cyclopropane-dicarboxylic acid, which was used “as is” for the next step, cyclization to the imide. A quantitative yield from the diester was assumed for calculation purposes.

### Step 4: Synthesis and Recrystallization of 1-(3,4-dichlorophenyl)-3-azabicyclo[3.1.0]hexane-2,4-dione

The slurry of 1-(3,4-dichlorophenyl)-1,2-cyclopropane-dicarboxylic acid (from Step 3) was added to 45.6 kg warm (68° C.) formamide, and residual ethyl acetate was distilled with full vacuum at 68-73° C. An additional 14.4 kg formamide was added to the mixture, followed by 11.2 kg of the dicarboxylic acid (derived from the disodium salt, Step 3). An argon blanket on the mixture was maintained for the following operation. The mixture was agitated 15 minutes at 73-75° C. to effect a complete solution, and then heated over a 1 hour period to 140-145° C. and maintained at this temperature for an additional 2.25 hours. Heating was discontinued, and the mixture was cooled to 70° C. and 10 L water containing 20 ml 32% HCl was slowly added over 30 minutes. The mixture was seeded and crystallization commenced. An additional 20 L water was slowly added to the heavy suspension over a 2 hour period. After standing overnight at ambient conditions, the mixture was agitated for 1.25 hours at ambient temperature and then suction-filtered (Nutsche). The cake of crude 1-(3,4-dichlorophenyl)-3-azabicyclo-[3.1.0]hexane-2,4-dione was washed with water (3×20 L), suction dried for 30 minutes and air-dried on paper (racks) for 2 days under ambient conditions.

A mixture of 37 kg crude, damp 1-(3,4-dichlorophenyl)-3-azabicyclo-[3.1.0]hexane-2,4-dione (from Step 4, above) and 120 L toluene was warmed to 75-80° C. to effect solution. After stratification and separation of the residual water (3.3 kg), 1 kg Darco G-60 activated carbon (American Norit Co.) (suspended in 5 L toluene) was added. The mixture was agitated at 80° C. for 30 minutes and then pressure filtered through a preheated Sparkler (precoated with filteraid), polishing with a 10 μm in-line filter. The clear light yellow solution was concentrated in vacuo at 75-80° C. to 100 L final volume and slowly cooled, with seeding at 70° C. The heavy crystalline suspension was cooled to −5° C., held 30 minutes at this temperature and suction-filtered (Nutsche). The cake of purified 1-(3,4-dichlorophenyl)-3-azabicyclo-[3.1.0]hexane-2,4-dione was washed with 2×10 L cold (−10° C.) toluene, and then 2×20 L hexane. After suction drying for 30 minutes, the 2,4-dione product was dried in vacuo (≦62° C.).

### Step 5: Synthesis and Purification of (±)-1-(3,4-Dichlorophenyl)-3-azabicyclo[3.1.0]hexane hydrochloride

BH3-THF complex is charged into a 2 L addition funnel (9×2 L, then 1×1.5 L) and drained into a 50 L flask.

1000 g of (±)-1-(3,4 dichlorophenyl)-3-azabicyclo[3.1.0]-hexane-2,4-dione is dissolved in 2 L of THF and added to the BH3-THF dropwise over a period of 2 hours. The reaction mixture is heated to reflux and held at this temperature overnight. The mixture is then cooled to <10° C., adjusted to pH 2 with the addition of 1200 mL of 6N HCl dropwise at <20° C., and stirred for a minimum of 1 hour.

The reaction mixture is then transferred to a 10 L Buchi flask, concentrated to a milky white paste, and transferred again to a 5-gallon container. The mixture is diluted with 4 L of cold water and adjusted to pH 10 with 2000 mL of a 25% sodium hydroxide solution. A temperature of <20° C. is maintained. Following this, 4.5 L of ethyl acetate is added and the mixture is stirred for 15 minutes. The solution is then filtered through a 10 inch funnel with a filter cloth and washed with ethyl acetate (2×250 mL).

The filtrate is then transferred into a 40 L separatory funnel and the phases are allowed to separate. Each phase is then drained into separate 5-gallon containers. The aqueous layer is returned to the 40 L separatory funnel and extracted with ethyl acetate (2×2 L). The organic phases are combined. The aqueous layer is discarded.

250 g of magnesium sulfate and 250 g of charcoal are added to the combined organics and the mixture is stirred well. The solution is then filtered through an 18.5 cm funnel using a filter pad and washed with ethyl acetate (2×250 mL). The filtrate is then transferred to a 10 L Buchi flask and concentrated to dryness. The resulting yellowish oil is diluted with ethyl acetate (2.25 mL/g).

HCl gas is bubbled through a 12 L flask containing 10 L of ethyl acetate to make an approximately 2.3 M solution of HCl/ethyL acetate. This HCl/ethyl acetate solution is added to the oil dropwise at a rate that maintains a temperature of <20° C. using an ice/water bath. The solution is then stirred at <10° C. for a minimum of 2 hours in the ice/water bath. The material is chilled in a cold room overnight.

The resulting solids are then filtered through a 10 inch funnel utilizing a filter cloth and washed with ethyl acetate (2×200 mL) and ethyl ether (3×500 mL). The product, crude (±)-1-(3,4-Dichlorophenyl)-3-azabicyclo[3.1.0]-hexane hydrochloride, is then transferred to Pyrex drying trays and dried for 4 hours. 1900 g of crude (+)-1-(3,4-dichlorophenyl)-3-azabicyclo[3.1.0]hexane hydrochloride from above, and 15.2 L of isopropyl alcohol are charged to a 22 L flask. The mixture is heated to dissolve all material.

The material is then filtered through a 18.5 cm funnel utilizing a filter pad and transferred to a 22 L flask. The solution is then stirred at room temperature for 1 hour. After stirring, the solution is chilled to 4° C. with an ice/water bath and stirred for 3.75 hours. The product is then placed in a cold room overnight.

The solids are then filtered through a 13 inch filter using a filter cloth and washed with ethyl ether (3×633 mL). The product is then air dried for 2 hours.

The product, pure (±)-1-(3,4-dichlorophenyl)-3-azabicyclo[3.1.0]hexane hydrochloride, is transferred to clean Pyrex drying trays and dried to constant weight.

### Step 6: Resolution of (±)-1-(S3,4-dichlorophenyl)-3-azabicyclo[3.1.0]hexane hydrochloride into (+)-1-(3,4-dichlorophenyl)-3-azabicyclo[3.1.0]hexane hydrochloride

In a 50 gallon reactor containing 60 L of 15% NaOH, 13.6 kg of pure (±)-1-(3,4dichlorophenyl)-3-azabicyclo[3.1.0]hexane hydrochloride (from Step 5, above) is added while keeping the temperature constant at approximately 20° C. Once the addition of (±)-1-(3,4-dichlorophenyl)-3-azabicyclo[3.1.0]hexane hydrochloride is complete, the reaction mixture is allowed to stir at room temperature for a minimum of 8 hours.

40 L of ethyl acetate is added to the reactor and the two phase mixture is stirred until a clear solution is obtained (approximately 2 hours). The phases are allowed to separate and the organic layer is transferred to another 50 gallon reactor. The remaining aqueous layer is extracted with ethyl acetate (6×6 L). All organic phases are combined into the 50-gallon reactor. The organic phase is dried and decolorized by the addition of 4000 g magnesium sulfate and 250 g of charcoal. The mixture is then filtered through an in-line filter. The filtrate is transferred via in-line filter to a 50-gallon reactor.

In a separate 50-gallon reactor, 23,230 g of L-(−)-dibenzoyl tartaric acid is dissolved with stirring (approximately 30 minutes) in 71 L of methanol. The dissolution is assisted with heating if necessary.

The L-(−)-dibenzoyl tartaric acid solution in methanol is added via addition funnel to the reactor containing the filtrate, over a period of approximately 1 hour, maintaining the temperature at 15-25° C. After the addition is complete the mixture is stirred for approximately 16 hours at 15-25° C. Following stirring, 50 L of methanol is added to the mixture and it is stirred again for 30 additional minutes. The resulting solids are filtered onto a plate filter. The solids are then washed with methanol (3×5 L) and pressed dry. The crude solids are weighed and transferred to a 50-gallon reactor to which 80 L of methanol is added. The mixture is heated to reflux and stirred at reflux for approximately 30 minutes. The mixture is then cooled to 15-20° C. and stirred at this temperature for approximately 2 hours. The resulting solids are filtered onto a plate filter using a polypropylene filter cloth. The cake is washed with methanol (3×5 L) and pressed dry. The solids are transferred to a tarred 5-gallon container and weighed (yield ˜20 kg).

The solids are then added (over a period of approximately 1 hour) to a 50 gallon reactor vessel containing 60 L of 15% NaOH while maintaining the temperature at approximately 20° C. Once the addition of the solids is complete the reaction mixture is stirred for approximately 19 hours.

40 L of ethyl acetate is charged to the reactor, while maintaining the temperature at ≦35° C. and the two phase mixture is stirred until a clear solution is obtained (approximately 2 hours). The phases are allowed to separate and the organic layer is transferred to another 50 gallon reactor. The remaining aqueous layer is extracted with ethyl acetate (6×6 L). All organic phases are combined into the 50-gallon reactor. 5000 g of magnesium sulfate is then added to the organic phase. The mixture is then filtered through an in-line filter. The filtrate is transferred via in-line filter to a 50-gallon reactor. The filtrate is concentrated to a total volume of 20-30 L.

In a 22 L three neck round bottom flask, HCl gas is bubbled through 12 L of ethyl acetate to make an approximately 2.3 M solution of HCl/ethyl acetate. After titration assay, the solution is adjusted to exactly 2.3 M by adding either ethyl acetate or HCl gas.

8.2 L of the 2.3 M solution of HCl/ethyl acetate is added (over a period of approx. 1.5 hours) to the filtrate (above), maintaining the temperature at ≦20° C. and ensuring that a pH of 2 is obtained. Once the addition is complete, the mixture is stirred at 0 to −5° C. for a period of 16 hours.

The resulting solids, crude (+)-1-(3,4-dichlorophenyl)-3-azabicyclo[3.1.0]hexane hydrochloride, are filtered onto a plate filter using a polypropylene filter cloth. The solids are then washed with ethyl acetate (2×2 L), acetone (2×2 L) and ethyl ether (2×2 L) and dried under vacuum. The material is transferred to a tarred 5-gallon polyethylene container and weighed.

### Step 6a: Recrystallization of (+)-1-(3,4-dichlorophenyl)-3-azabicyclo[3.1.0]hexane hydrochloride from isopropanol

The solids (from Step 6, above) are transferred to a 50-gallon reactor and isopropanol is added (8-10 mL/g of solid). The mixture is heated to reflux. The solution is filtered through an in-line filter into another 50 gallon reactor. The solution is cooled to 0 to −5° C. and maintained at this temperature with stirring for approximately 2 hours. The resulting solids are filtered onto a plate filter using a polypropylene filter cloth. The solids are then washed with ethyl acetate (2×2 L), acetone (2×2 L) and ethyl ether (2×2 L). The solids are dried under vacuum.

The product, (+)-1-(3,4-dichlorophenyl)-3-azabicyclo[3.1.0]hexane hydrochloride, is transferred into clean, tarred drying tray(s). The tray(s) are placed in a clean, vacuum drying oven. The product is dried at 50° C. to constant weight. The material is dried for a minimum of 12 hours at <10 mm Hg. This product was a mixture of polymorph form A and polymorph form B, with each polymorph present in the mixture in an amount of about 50% by weight. This product was used as the starting material for Examples V, VI, and VII below.

### Example V

### Preparation of Polymorph Form A of (+)-1-(3,4-dichlorophenyl)-3-azabicyclo[3.1.0]hexane

As in U.S. patent application Ser. No. 12/428,399, 20 mg samples of the 50% by weight mixture of polymorph form A and polymorph form B of the hydrochloride salt of (+)-1-(3,4-dichlorophenyl)-3-azabicyclo[3.1.0]hexane from Example IV were dissolved in 0.5 ml of aqueous ethanol. Other samples were prepared by dissolving 20 mg of this mixture in 0.5 mL of water. Both solutions were filtered through a 0.2 micron nylon filter. Both filtered solutions were then allowed to evaporate under ambient conditions, some samples partially covered and other samples completely uncovered. After 6 days, both the uncovered and partially covered ethanol solution samples evaporated. After 7 days, the uncovered water solutions evaporated. After 15 days, the partially covered water solutions evaporated. For each sample, after the solvent (either aqueous ethanol or water) evaporated completely, 20 mg of dry solid residue was left. The solid in all samples thus produced was the pure polymorph form A crystals of the hydrochloride salt of (+)-1-(3,4-dichlorophenyl)-3-azabicyclo[3.1.0]hexane as demonstrated by Raman spectroscopy and XRPD analysis as described above.

### Example VI

### Preparation of Polymorph Form B of (+)-1-(3,4-dichlorophenyl)-3-azabicyclo[3.1.0]hexane

As in U.S. patent application Ser. No. 12/428,399, 40 mg samples of the 50% by weight mixture of polymorph form A and polymorph form B of the hydrochloride salt of (+)-1-(3,4-dichlorophenyl)-3-azabicyclo[3.1.0]hexane from Example IV were mixed with 0.5 mL of anhydrous acetonitrile to produce a concentration of about 80-100 mg/mL and the resulting samples were stirred at various temperatures between 50° C. and 80° C. for various periods of time (some for 4 days and 6 days at about 50° C. and some for 1 day at about 80° C.). The resulting samples were each mixtures of a clear liquid and some solid. The clear liquid was decanted off, and the remaining solid was vacuum dried at ambient temperature for 1 hour to 2 days (50° C. sample), or 6 days (80° C. sample) to afford pure crystalline polymorph form B. All samples produced the pure polymorph form B crystals of the hydrochloride salt of (+)-1-(3,4-dichlorophenyl)-3-azabicyclo[3.1.0]hexane as demonstrated by Raman spectroscopy and XRPD analysis as described above.

### Example VII

### Preparation of Polymorph Form C of (+)-1-(3,4-dichlorophenyl)-3-azabicyclo[3.1.0]hexane

51 mg of the 50% by weight mixture of polymorph form A and polymorph form B of the hydrochloride salt of (+)-1-(3,4-dichlorophenyl)-3-azabicyclo[3.1.0]hexane prepared in Example IV was weighed into a vial. The vial was covered with aluminum foil perforated with pinholes and placed in an oven at 80° C. for 4 days to produce the pure polymorph C crystals of the hydrochloride salt of (+)-1-(3,4-dichlorophenyl)-3-azabicyclo[3.1.0]hexane as demonstrated by Raman spectroscopy and XRPD analysis as described above.

### Example VIII

### Activity Comparison of (+)-1-(3,4-dichlorophenyl)-3-Azabicyclo[3.1.0]Hexane and (±)-1-(3,4-dichlorophenyl)-3-Azabicyclo[3.1.0]Hexane

### Norepinephrine Transporter Binding Assay

The norepinephrine binding assay was performed according to the methods described in Raisman et al., Eur. J. Pharmacol. 78:345-351 (1982) and Langer et al., Eur. J. Pharmacol. 72:423 (1981). The receptor source was rat forebrain membranes; the radioligand was [³H]-nisoxetine (60-85 Ci/mmol) at a final ligand concentration of 1.0 nM; the non-specific determinant [1.0 μm]; reference compound and positive control were (±)-desmethylimipramine HCl. (+)-1-(3,4-dichlorophenyl)-3-azabicyclo[3.1.0]hexane HCl was obtained according to the method of Example 1, above. Reactions were carried out in 50 mM TRIS-HCl (pH 7.4), containing 300 mM NaCl and 5 mM KCl at 0° C. to 4° C. for 4 hours. The reaction was terminated by rapid vacuum filtration onto glass fiber filters. Radioactivity trapped in the filters was determined and compared to control values in order to ascertain the interactions of the test compound with the norepinephrine uptake site. The data are reported in Table 5 below.

TABLE 5
Norepinephrine Transporter Binding Assay

| Compound | Ki |
|---|---|
| (±)-1-(3,4-dichlorophenyl)-3-Azabicyclo[3.1.0]Hexane | 1.42 × 10⁻⁷ |
| (+)-1-(3,4-dichlorophenyl)-3-azabicyclo[3.1.0]hexane HCl | 8.20 × 10⁻⁸ |
| (±)-desmethylimiprimine HCl | 1.13 × 10⁻⁹ |

The data in Table 5 show that (+)-1-(3,4-dichlorophenyl)-3-azabicyclo[3.1.0]hexane HCl has a significantly greater affinity for the norepinephrine uptake site than does the (±)-1-(3,4-dichlorophenyl)-3-azabicyclo[3.1.0]hexane HCl. Successful inhibition of norepinephrine reuptake has been has been associated with the treatment of one or more of the symptoms of depression (R. J. Baldessarini, Drugs and the Treatment of Psychiatric Disorders: Depression and Mania, in Goodman& Gilman's The Pharmacological Basis of Therapeutics 431-459 (9^{th} ed. 1996)). Therefore, (+)-1-(3,4-dichlorophenyl)-3-azabicyclo[3.1.0]hexane or a pharmaceutically acceptable salt thereof will be significantly more active than (±)-1-(3,4-dichlorophenyl)-3-azabicyclo[3.1.0]hexane or a pharmaceutically acceptable salt thereof for treating or preventing depression in a patient.

### Serotonin Transporter Binding Assay

The serotonin binding assay was performed according to the methods described in D'Amato et al., J. Pharmacol. Exp. Ther. 242:364-371 (1987) and Brown et al., Eur. J. Pharmac. 123:161-165 (1986). The receptor source was rat forebrain membranes; the radioligand was [³H]-citalopram (70-87 Ci/mmol) with a final ligand concentration of 0.7 nM; the non-specific determinant was clomipramine [10 μm]; and the reference compound and positive control were (±)-desmethylimipramine. (+)-1-(3,4-dichlorophenyl)-3-azabicyclo[3.1.0]hexane HCl was obtained according to the method of Example 5, above. Reactions were carried out in 50 mM TRIS-HCl (pH 7.4) containing 120 mM NaCl and 5 mM KCl at 25° C. for 60 minutes. The reaction was terminated by rapid vacuum filtration onto glass fiber filters. Radioactivity trapped in the filters was determined using liquid scintillation spectrometry and compared to control values in order to ascertain any interactions of test compound with the serotonin transporter binding site. The data are reported in Table 6 below.

TABLE 6
Serotonin Transporter Binding Assay

| Compound | Ki |
|---|---|
| (±)-1-(3,4-dichlorophenyl)-3-Azabicyclo[3.1.0]Hexane | 1.18 × 10⁻⁷ |
| (+)-1-(3,4-dichlorophenyl)-3-azabicyclo[3.1.0]hexane HCl | 5.08 × 10⁻⁸ |
| (±)-desmethylimiprimine HCl | 2.64 × 10⁻⁸ |

The data in Table 6 show that (+)-1-(3,4-dichlorophenyl)-3-azabicyclo[3.1.0]hexane HCl has a significantly greater affinity for the serotonin uptake site than does (±)-1-(3,4-dichlorophenyl)-3-azabicyclo[3.1.0]hexane HCl. Successful inhibition of serotonin reuptake has been has been associated with the treatment of one or more of the symptoms of depression (R. J. Baldessarini, Drugs and the Treatment of Psychiatric Disorders: Depression and Mania, in Goodman& Gilman's The Pharmacological Basis of Therapeutics 431-459 (9^{th} ed. 1996)). Therefore, (+)-1-(3,4-dichlorophenyl)-3-azabicyclo[3.1.0]hexane or a pharmaceutically acceptable salt thereof will be significantly more active than (±)-1-(3,4-dichlorophenyl)-3-azabicyclo[3.1.0]hexane or a pharmaceutical salt thereof for treating or preventing depression in a patient. (See U.S. Pat. No. 6,372,919)

### Example IX

### Efficacy of (+)-1-(3,4-dichlorophenyl)-3-azabicyclo[3.1.0]hexane in the Treatment of patients with Major Depressive Disorder

Subjects were identified who were between the ages of 18-65 (inclusive), and met criteria for Major Depressive Disorder in accordance with the Diagnostic and Statistical manual of Mental Disorders-IV-TR and confirmed by the MINI International Neuropsychiatric Interview. At the screening visit, subjects had a baseline Hamilton Depression Rating Scale (HAMD-17)≧22 and a severity of ≧2 on item 1 and a rating on the Hamilton Anxiety Scale (HAM-A)<17. They were also required to have a BMI ≦35 and body weight >45 kg at the Screening Visit.

They were excluded if they were judged to be a suicide risk, known to be antidepressant treatment resistant or had other major clinically significant medical and/or other psychiatric illnesses such as panic disorder, social phobia, generalized anxiety disorder, obsessive compulsive disorder, post-traumatic stress disorder, acute stress disorder, substance abuse, anorexia, bulimia, antisocial personality disorder or bipolar disorder. Additionally, subjects who had a HAMD-17 reduction in score of more than 15% between the Placebo run-in visit and the baseline visit were eliminated.

Subjects were required to refrain from taking antidepressants, anticonvulsants including gabapentin and pregabalin, neuroleptics, MAO inhibitors, barbiturates, benzodiazepines, stimulants, antipsychotics, lithium, anxiolytics and beta blockers starting two weeks prior to the study and continuing until after the follow-up visit.

Subjects were evaluated for safety parameters prior to and throughout the trial by a variety of measures including electrocardiogram, physical examination, vital signs and body weight, and clinical laboratory testing including a lipid panel, CBC with differential and urinalysis, Samples were drawn to assess total bilirubin, alkaline phosphatase, ALT (SGPT), AST (SGOT), blood urea nitrogen (BUN), creatinine, glucose, uric acid, calcium, phosphorus, total protein, albumin, total cholesterol, LDL, HDL, triglycerides, sodium, potassium, bicarbonate, chloride, GGT and creatine kinase, Hepatitis B, C and HIV serologies, TSH, drug screen and serum pregnancy test for females. Sixty-three eligible subjects were identified who were not eliminated by the safety parameters. These sixty-three subjects had the following combined (placebo and EB-1010) mean baseline scores on the main outcome measures: MADRS (31.4) (primary); HAMD-17 (29.6) (secondary); and DISF-SR (25.38). The sixty-three subjects were randomized to receive either 25 mg of (±)-1-(3,4-dichlorophenyl)-3-azabicyclo[3.1.0]hexane twice a day for two weeks and then 50 mg twice a day for four weeks or placebo according to the following schedule:

TABLE 7
Titration schedule

| | | Study Medication Dispense Visit | | | | | |
|---|---|---|---|---|---|---|---|
| | | | | | | . . . | Visit 7 |
| | | Visit 3: | Visit 4 | Visit 5 | Visit 6 | (Day 29 ± 2) | Visit 8/EOT |
| | | Baseline/Day 1 | (Day 8 ± 2) | (Day 15 ± 2) | (Day 22 ± 2) | (Visit 7-1 and | (Day 43 ± 2) |
| Study Groups | | (Visit 3 Blister) | (Visit 4 Blister) | (Visit 5 Blister) | (Visit 6 Blister) | Visit 7-2 Blisters) | (Visit 8 Blister) |
| Placebo | Morning | 2 Placebo Capsules | 2 Placebo Capsules | 2 Placebo Capsules | 2 Placebo apsules | 2 Placebo Capsules | 2 Placebo Capsules |
| | Dose | | | | | | |
| | Evening | 2 Placebo Capsules | 2 Placebo Capsules | 2 Placebo Capsules | 2 Placebo Capsules | 2 Placebo Capsules | 2 Placebo Capsules |
| | Dose | | | | | | |
| DOV | Morning | 25 mg Capsule: 1 | 25 mg Capsule: 1 | 25 mg Capsule: 2 | 25 mg Capsule: 2 | 25 mg Capsule: 2 | 2 Placebo Capsules |
| 21,947 | Dose | Placebo Capsule: 1 | Placebo Capsule: 1 | | | | |
| | Evening | 25 mg Capsule: 1 | 25 mg Capsule: 1 | 25 mg Capsule: 2 | 25 mg Capsule: 2 | 25 mg Capsule: 2 | 2 Placebo Capsules |
| | Dose | Placebo Capsule: 1 | Placebo Capsule: 1 | | | | |

Visits and evaluations were performed according to the following schedule of events:

Visit 1: Screening Visit:

The following was obtained/performed at the Screening Visit (Visit 1):
Written informed consent
Medical history including:
   Relevant demographic information
   Detailed medical and surgical history, including review of systems

Whenever possible, the patient's medical history was confirmed by medical records.
Prior medication: Medication taken by the patients 30 days prior to the Screening Visit was recorded.
AE assessment
Height (cm)
Weight (kg); BMI was determined and was ≦35 for the patient to be randomized
Complete physical examination
MINI diagnostic exam
Vital signs (respiratory rate, oral temperature (° C.), blood pressure, pulse). Blood pressure and pulse was measured twice: supine, after resting supine for at least 5 min and then at least 2 min but less than 3 min after standing up.
Fasted clinical laboratory tests (chemistry, CBC with differential and urinalysis)
Hepatitis B, C and HIV serologies, TSH
Resting 12-lead ECG
Urine drug screen
Pregnancy test (females; serum)
Review of inclusion and exclusion criteria
HAM-A (a score <17 is required for enrollment)

Visit 2: Placebo Run-In Visit:

The following procedures were performed:
Concomitant medication record
AE assessment
Review inclusion and exclusion criteria
HAMD-17: To be eligible for the study, the total HAMD-17 score must be ≧22 and the score on HAMD-17 item 1 must be ≧2.
Patients found to be eligible were dispensed a single blind placebo blister package (the Visit 2 blister). The capsules were taken for 7 days prior to the Baseline/Day 1 Visit (Visit 3). The first dose of placebo was taken at the clinic with 240 mL of water after a light meal.
Patients were provided with a diary to record the date, time and dosage of each dose.

Patient Medication Diary:

Patients were provided with a diary at the Placebo Run-In Visit (Visit 2) and at each subsequent visit except the last visit (the Follow-Up Visit, Visit 9). Patients recorded the date, time and dosage of each study medication dose using the diary. The diary was collected at the next scheduled visit, reviewed for dosing compliance, and a new diary dispensed.

TABLE 8
Schedule of Events After Screening

| | Visit 3/ | Visit 4/ | Visit 5/ | Visit 6/ | Visit 7/ | Visit 8/ | Visit 9/ |
|---|---|---|---|---|---|---|---|
| Procedure | Baseline | Week 2 | Week 3 | Week 4 | Week 5 | Week 6 | Post Treatment |
| Day | | 8 ± 2 | 15 ± 2 | 22 ± 2 | 29 ± 2 | 43 ± 2 | 50 ± 2 |
| Vital Signs | X | | | | | | |
| Height | | | | | | X | |
| Weight | | | | | | X | |
| 12-lead ECG | X | | | | | | |
| Physical | X | X | X | X | X | X | X |
| Examination | | | | | | | |
| Concomitant | X | X | X | X | X | X | X |
| Medication | | | | | | | |
| Inclusion/ | X | | | | | | |
| Exclusion Criteria | | | | | | | |
| Fasted Lab Work | X | | X | | X | X | X |
| | (and lipid | | | | | (and lipid | |
| | profile) | | | | | profile) | |
| Collect blood | X | | X | | X | | |
| sample | | | | | | | |
| Collect Urine | X | X | | X | | X | |
| Sample | | | | | | | |
| Urine Drug | X | | | | | | |
| Screen | | | | | | | |
| Serum Pregnancy | X | | | | | X | X |
| (females only) | | | | | | | |
| HAMD-17 | X | | | | | | |
| MADRS | X | | | | | | |
| DISF-SR | X | | | | | | |
| CGI-S | X | | | | | | |
| Review | X | | | | | | |
| Inclusion/Exclusion | | | | | | | |
| Criteria | | | | | | | |
| Adverse Event | X | X | X | X | X | X | |
| Assessment | | | | | | | |
| Medication | X | X | X | X | X | X | |
| Dispensed | | | | | | | |
| Collect Diary | X | X | X | X | X | X | |

Post Dose

| | | | | | | | |
|---|---|---|---|---|---|---|---|
| Vital Signs | X | X | X | X | X | X | X |
| (1.5 hours after | | | | | | | |
| dosing) | | | | | | | |
| ECG-12 Lead | X | | X | | X | X | X |
| HAMD-17 | | X | X | X | X | X | X |
| CGI-I | | X | X | X | X | X | X |
| CGI-S | | X | X | X | X | X | X |
| DISF-SR | | | X | | X | X | X |
| MADRAS | | X | X | X | X | X | X |

Efficacy was determined by measuring the change from baseline in the Montgomery-Åsberg Depression Rating Scale (MADRS), the HAMD-17, the Clinical Global Impression Global Improvement Scale (CGI-I), the Clinical Global Impression-Severity scale (CGI-S) and the Derogatis Interview for Sexual Functioning Self-Report (DISF-SR). Two analysis populations were studied: Modified Intent to Treat (MITT, N=56), defined as all randomized subjects with any confirmed dosing and MADRS data from at least one post-baseline visit (30 EB-1010-treated patients and 26 placebo-treated patients); and Completers (N=39), defined as the subset of MITT subjects who completed 6 weeks of treatment (20 EB-1010-treated patients and 19 placebo-treated patients). Comparisons between treatment groups based on MADRS (the primary efficacy parameter), HAMD-17, Anhedonia, DISF-SR, CGI-I and CGI-S scores were analyzed using a mixed-repeated measures (MMRM) analysis model including factors for Subject, Visit, Treatment Arm and Baseline value as a covariate. Adjusted least-squares means from these models are presented. Comparisons between groups were made at each post-baseline visit using model-based contrasts and adjusted degrees of freedom. For these analyses no explicit data imputations were made prior to the analysis. Response and remission categorical data were analyzed using chi-square tests. Inferential analyses of safety data were conducted with ANOVA models or chi-square tests. Two-tail alpha was set to 0.05. All analyses were conducted using SAS version 9.2.

The intent-to-treat (ITT) population (n=56) showed the following combined (placebo and (+)-1-(3,4-dichlorophenyl)-3-azabicyclo[3.1.0]hexane) mean baseline scores on the main outcome measures: MADRS (31.4) (primary); HAMD-17 (29.5) (secondary); and DISF-SR (25.8). As shown in FIG. 1, at the end of the double-blind treatment (Week 6), the estimated LS mean change from baseline (MMRM or mixed model repeated measures) in the MADRS total scores was statistically significantly superior for (+)-1-(3,4-dichlorophenyl)-3-azabicyclo[3.1.0]hexane when compared to placebo (18.16 vs 21.99; p=0.028), with an overall statistical effect size of −0.63 (Cohen's d). As shown in Table 9, when assessed with the CGI-I, a global impression scale sensitive to clinically relevant changes in improvement status, treatment with (+)-1-(3,4-dichlorophenyl)-3-azabicyclo[3.1.0]hexane was also statistically significantly superior to placebo (p=0.03; Week 6; MMRM). As shown in FIG. 6, an anhedonia factor score grouping Items 1 (apparent sadness), 2 (reported sadness), 6 (concentration difficulties), 7 (lassitude), and 8 (inability to feel) of the MADRS (analyzed using the mixed model for repeated measures LS means) demonstrated a statistically significant difference in favor of (+)-1-(3,4-dichlorophenyl)-3-azabicyclo[3.1.0]hexane in comparison to placebo (p=0.049). (+)-1-(3,4-dichlorophenyl)-3-azabicyclo[3.1.0]hexane was relatively well tolerated. Two patients in each treatment group discontinued the study early due to AEs but no serious AEs were reported.

TABLE 9
Least Square Adjusted Means with differences in Primary
and Secondary Efficacy Measures at Visit 8 (MMRM, MITT)

| | | (+)-1-(3,4-dichlorophenyl)- | | |
|---|---|---|---|---|
| | Placebo | 3-azabicyclo[3.1.0]hexane | Difference | |
| Outcome | (n = 26) | (n = 30) | (95% CI) | P value |
| MADRS (LS Mean-SE) | 21.99 (1.24) | 18.16 (1.21) | 3.83 (0.41, 7.26) | P = 0.028 |
| HAMD-17 (LS Mean-SE) | 18.02 (1.46) | 14.90 (1.40) | 3.12 (−0.87, 7.12) | P = 0.125 |
| Anhedonia factor (LS Mean- | 9.33 (0.50) | 7.92 (0.50) | 1.41 (0.01, 2.82) | P = 0.049 |
| SE) | | | | |
| CGI-I (LS Mean-SE) | 2.75 (0.20) | 2.13 (0.20) | 0.62 (0.06, 1.18) | P = 0.030 |
| CGI-S (LS Mean-SE) | 3.53 (0.15) | 3.31 (0.15) | 0.22 (−0.21, 0.66) | P = 0.306 |

Abbreviations:
MADRS, Montgomery Åsberg Depression Rating Scale;
HAMD-17, Hamilton Rating Scale for Depression;
CGI-I, Clinical Global Impressions - Improvement;
CGI-S, Clinical Global Impressions - Severity;
MMRM, Mixed Effect Models for Repeated Measures;
MITT, Modified Intent-to-treat;
CI, Confidence Interval,
SE, Standard Error.

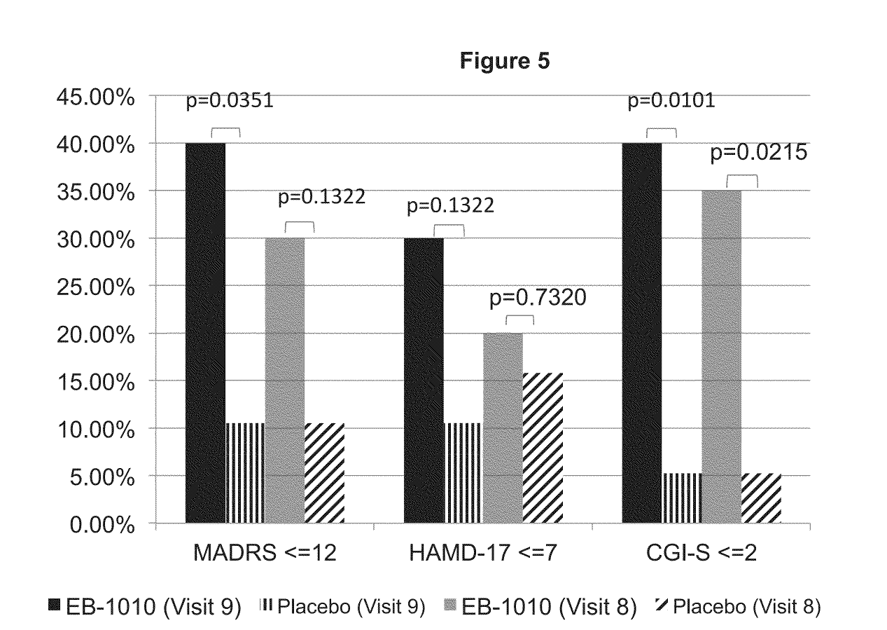

As shown in Table 10 and FIG. 5 (data analyzed using the last observation carried forward method), treatment with 100 mg of (+)-1-(3,4-dichlorophenyl)-3-azabicyclo[3.1.0]hexane was associated with significantly greater remission rates, defined by achieving a CGI-S score of ≦2, compared to placebo.

TABLE 10
Response and Remission Rates (Visit 8, LOCF, Completers)

| | (+)-1-(3,4-dichlorophenyl)-3- | | | |
|---|---|---|---|---|
| | azabicyclo[3.1.0]hexane 100 mg | Placebo | Odds Ratio | |
| Outcome | [n/N] (%) | [n/N] (%) | (95% CI) | P value |
| Response | | | | |
| MADRS | (8/20) 40.00% | (3/19) 15.79% | 0.281 (0.061, 1.290) | 0.093 |
| HAMD-17 | (11/20) 55.00% | (7/19) 36.84% | 0.477 (0.132, 1.721) | 0.256 |
| Remission | | | | |
| MADRS | (6/20) 30.00% | (2/19) 10.53% | 0.275 (0.048, 1.579) | 0.132 |
| HAMD-17 | (4/20) 20.00% | (3/19) 15.79% | 0.750 (0.144, 3.904) | 0.732 |
| CGI-S | (7/20) 35.00% | (1/19) 5.26% | 0.103 (0.011, 0.944) | 0.022 |

Abbreviations:
MADRS, Montgomery Åsberg Depression Rating Scale;
HAMD-17, Hamilton Rating Scale for Depression;
CGI-I, Clinical Global Impressions - Improvement;
LOCF, Last Observation Carried Forward;
Response, 50% reduction or more of the baseline total score of MADRS or HAMD-17 at endpoint;
Remission, MADRS ≦12 or HAMD-17 ≦7 or CGI-S ≦2.

Additionally, unlike many antidepressants, as shown in FIG. 7, the DISF-SR scores stratified by low mean baseline scores (<25, indicating poor sexual function at baseline) versus high mean baseline scores (≧25, indicating preserved sexual function at baseline). In both the low baseline and high baseline groups, there are no differences between (+)-1-(3,4-dichlorophenyl)-3-azabicyclo[3.1.0]hexane 100 mg and placebo, indicating that treatment with (+)-1-(3,4-dichlorophenyl)-3-azabicyclo[3.1.0]hexane is not associated with emergence of sexual dysfunction. The efficacy of treatment with (+)-1-(3,4-dichlorophenyl)-3-azabicyclo[3.1.0]hexane was observed on the primary and secondary standard validated depression outcome measures (MADRS; global severity and improvement) as well as on the anhedonia factor of the MADRS. Furthermore, as shown in Tables 11 and 12, treatment with (+)-1-(3,4-dichlorophenyl)-3-azabicyclo[3.1.0]hexane was well tolerated and did not result in significant increases in heart rate, systolic or diastolic blood pressure compared to placebo. The number and percentage of patients who reported an adverse treatment event was similar between the two treatment groups (10 or 30.30% for EB-1010 ((+)-1-(3,4-dichlorophenyl)-3-azabicyclo[3.1.0]hexane) versus 11 or 39.28% for placebo).

TABLE 11
Treatment-Emergent Adverse Events* (% of Patients)

| | | Placebo |
|---|---|---|
| | EB-1010 (n = 33) | (n = 28) |
| Headache NOS | 3 (9.09%) | 3 (10.71%) |
| Abdominal Pain (NOS) | 2 (6.06%) | 1 (3.57%) |
| Anxiety | 2 (6.06%) | 1 (3.57%) |
| Diarrhea NOS | 2 (6.06%) | 1 (3.57%) |
| Irritability | 2 (6.06%) | 1 (3.57%) |
| Nausea | 2 (6.06%) | 1 (3.57%) |
| Rash NOS | 2 (6.06%) | 1 (3.57%) |
| Upper Respiratory Tract Infection NOS | 2 (6.06%) | 1 (3.57%) |
| Emotional Disturbance NOS | 2 (6.06%) | 0 (0.00%) |

*Treatment-emergent adverse events defined as events reported by at least 5% of EB-1010-treated patients and at least twice the rate of placebo

TABLE 12
Changes From Baseline in Selected Vital Signs and
Laboratory Values at Visit 8, Safety Population (n = 61)

| | EB-1010 | Placebo | |
|---|---|---|---|
| | (n = 33) | (n = 28) | |
| | Mean | Mean | P value vs. |
| Assessment [Units] | Change | Change | placebo |
| Systolic BP - Supine [mmHg] | 2.58 | 2.28 | 0.904 |
| Diastolic BP - Supine [mmHg] | −0.38 | −0.48 | 0.961 |
| Systolic BP - Standing (mmHg) | 0.069 | 2.12 | 0.509 |
| Diastolic BP - Standing (mmHg) | −3.00 | 2.80 | 0.017 |
| Supine Pulse [beats per minute] | 1.55 | −1.68 | 0.145 |
| Weight [kg] | 0.078 | 0.04 | 0.965 |
| Total Cholesterol Fasting [mg/dL] | −5.86 | −11.36 | 0.412 |
| LDL Cholesterol Fasting [mg/dL] | −4.29 | −9.96 | 0.374 |
| Triglycerides Fasting [mg/dL] | −12.00 | −7.80 | 0.750 |

Abbreviations:
BP blood pressure;
HDL high density lipoprotein;
LDL, low density lipoprotein;
Safety population: All randomized patients who received study drug;
P values were calculated by using ANOVA with treatment group as main effect

Additionally, treatment with (+)-1-(3,4-dichlorophenyl)-3-azabicyclo[3.1.0]hexane was not associated with significant weight gain or sexual dysfunction (See, for example, FIG. 7).

The results of this Phase 2 study demonstrated that EB-1010, at a titrated dose of 50 mg/day then 100 mg/day, was effective for treatment of patients with MDD. Efficacy was observed on the primary and secondary standard validated depression outcome measures (MADRS; global severity and improvement) as well as on the anhedonia factor of the MADRS. Overall, treatment with EB-1010 was well tolerated. The discontinuation rate due to AE was similar to placebo and treatment with EB-1010 was not associated with weight gain or sexual dysfunction.

### Example X

### Preparation of 50 Mg. (+)-1-(3,4-dichlorophenyl)-3-azabicyclo[3.1.0]hexane HCl Tablet

Immediate release tablets containing 50 mg of the HCl salt of (+)-1-(3,4-dichlorophenyl)-3-azabicyclo[3.1.0]hexane are prepared using the following ingredients. In table 13 below the “% composition” is the % by weight of the ingredient based upon the total weight of the composition.

TABLE 13
(+)-1-(3,4-dichlorophenyl)-3-azabicyclo[3.1.0]hexane HCl Tablets

| Material | % Composition | Mg/tablet |
|---|---|---|
| (+)-1-(3,4-dichlorophenyl)-3- | 22.22 | 50.00 |
| azabicyclo[3.1.0]hexane (HCl salt) | | |
| Dibasic Calcium Phosphate, NF | 36.00 | 81.00 |
| Microcrystalline cellulose, NF | 36.00 | 81.00 |
| Croscarmellose Sodium, NF | 4.44 | 10.00 |
| Colloidal Silicon Dioxide, NF | 0.67 | 1.50 |
| Magnesium Stearate, NF (veg grade) | 0.67 | 1.50 |

Each tablet may also be coated with 6.00 mg of Opadry II White (85F18422).

### Example XI

### Preparation of 50 Mg. (+)-1-(3,4-dichlorophenyl)-3-azabicyclo[3.1.0]hexane HCl Capsule

Immediate release capsules containing 50 mg of the HCl salt of (+)-1-(3,4-dichlorophenyl)-3-azabicyclo[3.1.0]hexane are prepared using the following ingredients. In table 14 below the “% composition” is the % by weight of the ingredient based upon the total weight of the composition.

TABLE 14
(+)-1-(3,4-dichlorophenyl)-3-azabicyclo[3.1.0]hexane HCl Capsules

| Material | % Composition | Mg/tablet |
|---|---|---|
| (+)-1-(3,4-dichlorophenyl)-3- | 24.39 | 50.00 |
| azabicyclo[3.1.0]hexane (HCl salt) | | |
| Mannitol, Spray Dried, USP | 72.28 | 148.16 |
| Talc, USP | 2.63 | 5.40 |
| Magnesium Stearate, NF | 0.70 | 1.44 |

The ingredients are encapsulated in a white opaque capsule #3.

### Example XII

### Preparation of 100 Mg. (+)-1-(3,4-dichlorophenyl)-3-azabicyclo[3.1.0]hexane HCl Extended Release Tablet

Once per day, extended release tablets containing 100 mg of the HCl salt of (+)-1-(3,4-dichlorophenyl)-3-azabicyclo[3.1.0]hexane are prepared using the following ingredients. In table 15 below the “% composition” is the % by weight of the ingredient based upon the total weight of the composition.

TABLE 15
(+)-1-(3,4-dichlorophenyl)-3-azabicyclo[3.1.0]hexane
HCl Extended Release Tablets

| Material | % Composition | Mg/tablet |
|---|---|---|
| (+)-1-(3,4-dichlorophenyl)-3- | 28.6 | 100.00 |
| azabicyclo[3.1.0]hexane (HCl salt) | | |
| Methocel Premium CR | 30.0 | 105.00 |
| MicroCrystalline Cellulose | 20.4 | 71.50 |
| Starch 1500 | 20.0 | 70.00 |
| Colloidal Silicon Dioxide | 0.5 | 1.75 |
| Magnesium Stearate | 0.5 | 1.75 |

The tablets are manufactured by direct compression into ⅜″ round, standard biconvex tablets. The microcrystalline cellulose used is 90 micron grade. A pregelatinized starch is used in the tablets. The Methocel Premium CR can be Methocel K4 or Methocel K100. Each tablet may also be coated, such as with 5.5% Opadry II White (85F18422).

### Example XIII

### Dissolution of 100 Mg. (+)-1-(3,4-dichlorophenyl)-3-azabicyclo[3.1.0]hexane HCl Extended Release Tablet

Dissolution testing of tablets manufactured according to Example XII was performed on tablets containing either Methocel K4 or K100, and tablets were either coated or uncoated. Dissolution Testing was performed using USP Apparatus 2, 50 rpm, 900 ml water, 37° C.

TABLE 16
Dissolution Testing of (+)-1-(3,4-dichlorophenyl)-3-
azabicyclo[3.1.0]hexane HCl Extended Release Tablets

| Time | K4M | K4M | K100M | K100M |
|---|---|---|---|---|
| (Mins). | uncoated | coated | uncoated | coated |
| 30 | 11.11 | 0.26 | 10.13 | 0.38 |
| 60 | 16.77 | 0.30 | 14.92 | 0.20 |
| 120 | 23.79 | 1.78 | 22.71 | 0.38 |
| 240 | 35.35 | 9.36 | 34.98 | 1.80 |
| 360 | 43.14 | 19.91 | 45.49 | 6.66 |
| 480 | 52.24 | 30.95 | 53.30 | 14.39 |
| 600 | 59.22 | 40.32 | 59.99 | 23.27 |
| 720 | 67.67 | 49.85 | 66.98 | 32.78 |
| 1500 | 104.44 | 83.32 | 78.31 | 68.43 |

The results of the dissolution testing confirm that a slow dissolution profile was achieved for an extended release tablet of (+)-1-(3,4-dichlorophenyl)-3-azabicyclo[3.1.0]hexane, HCl salt form. The results further show that the (+)-1-(3,4-dichlorophenyl)-3-azabicyclo[3.1.0]hexane was released at or nearly at a continuous or nearly same rate over 24 hours, and in particular was released at a continual or nearly continual/same rate between 2-12 hours (120-720 minutes). The amount of (+)-1-(3,4-dichlorophenyl)-3-azabicyclo[3.1.0]hexane released over 24 hours was from about 65% (68% in the K100M coated example) to 100%, and overall averaged about 83% released, with 3 samples of tablets having released 78, 83., and 100% of the (+)-1-(3,4-dichlorophenyl)-3-azabicyclo[3.1.0]hexane initially contained therein. The amount of (+)-1-(3,4-dichlorophenyl)-3-azabicyclo[3.1.0]hexane released after 12 hours following administration was from about 55% to about 70%.

All publications and patents cited herein are incorporated herein by reference for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

“Depression”. World Health Organization. WHO.
Basile, A. S., et al., J. Pharmacol. Exp. Ther., 321:1208-1225 (2007)
Basile, A.; Janowsky, A.; Golembiowska, K.; Kowalska, M.; Tam, E.; Benveniste, M.; Popik, P.; Nikiforuk, A. et al. (2007). “Characterization of the antinociceptive actions of bicifadine in models of acute, persistent, and chronic pain”. The Journal of pharmacology and experimental therapeutics 321 (3): 1208-1225.
Beck, A. T., R. A Steer (1988) Manual for Beck Hopelessness Scale. Psychological Corp., Harcourt Brace Jovanovich San Antonio, Tex.
Beck, A. T., R. A Steer (1991) Manual for Beck Scale For Suicide Ideation. San Antonio, Tex.: Psychological Corporation.
Briley, M., Hum. Psychopharmacol. Clin. Exp. 19:S21-S25 (2004) [0449] Skolnick, P. in “Dopamine and glutamate in psychiatric disorders,” W. Schmidt, Editor; Humana Press, Totowa, Chapter 9, pp. 199-214 (2005) [0450] Atkinson, J. H. et al., Pain 83:137-145 (1999)
Chandler G M, Iosifescu D V, Pollack M H, Targum S D, Fava M Validation of the Massachusetts General Hospital Antidepressant Treatment History Questionnaire (ATRQ) CNS Neurosci Ther. 2009 Sep. 21. [Epub ahead of print]
Coric, Vladimir•Stock, Elyse G Pultz, Joseph•Marcus, Ronald•Sheehan, David V. Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Eshleman, A. J. et al., Journal of Pharmacology & Experimental Therapeutics 289:877-885 (1999)
Fava M, Davidson K G. Definition and epidemiology of treatment-resistant depression. Psychiatr Clin North Am. 1996; 19:179-200
Fava M, Evins A E, Dorer D J, Schoenfeld D A. The problem of the placebo response in clinical trials for psychiatric disorders: culprits, possible remedies, and a novel study design approach. Psychother Psychosom. 2003; 72(3):115-27
Fava M, Iosifescu D V, Pedrelli P, Baer L. Reliability and validity of the Massachusetts general hospital cognitive and physical functioning questionnaire. Psychother Psychosom. 2009; 78(2):91-7
Fava M, Rush A J, Thase M E, Clayton A, Stahl S M, Pradko J F, Johnston J A. 15 years of clinical experience with bupropion HCl: from bupropion to bupropion SR to bupropion XL. Prim Care Companion J Clin Psychiatry. 2005; 7(3):106-13
Fava M. Diagnosis and definition of treatment-resistant depression. Biol Psychiatry. 2003; 53(8):649-59
Fava M. Prospective studies of adverse events related to antidepressant discontinuation. J Clin Psychiatry. 2006; 67 Suppl 4:14-21
First, Michael B., Williams, Janet B. W., Spitzer, Robert L., and Gibbon, Miriam: Structured Clinical Interview for DSM-IV-TR Axis I Disorders, Clinical Trials Version (SCID-CT). New York: Biometrics Research, New York State Psychiatric Institute, 2007.
Frantz S. Drug discovery: playing dirty. Nature. 2005; 437:942-3.
Graff, Ole et al. Results of two double blind Placebo and Active-controlled Studies of GSK372475, a Triple Monoamine Reuptake Inhibitor, in the Treatment of Major Depressive Disorder. (ACNP 2009)
Gardner, E.; Liu, X.; Paredes, W.; Giordano, A.; Spector, J.; Lepore, M.; Wu, K.; Froimowitz, M. (2006). “A slow-onset, long-duration indanamine monoamine reuptake inhibitor as a potential maintenance pharmacotherapy for psychostimulant abuse: effects in laboratory rat models relating to addiction”. Neuropharmacology 51 (5): 993-1003.
Green, T. W. and Wuts, P. G. M. in “Protective Groups in Organic Chemistry”, 3rd edition, John Wiley & Sons, New York, N.Y., 1999
Gu, H., et al. J. Biol. Chem. 269:7124-7130 (1994)
Guy W. Clinician Global Impression (CGI). ECDEU Assessment Manual for Psychopharmacology. 1976. Rockville, Md., U.S. Department of Health, Education, and Welfare.
Hamilton M. A rating scale for depression. J. Neurol. Neurosurg. Psychiat., 1960, 23, 56.
Hamilton M. Development of a rating scale for primary depressive illness. Br J Soc Clin Psychol. 1967; 6(4):278-96
Hamilton, M. (1959). The assessment of anxiety states by rating. British Journal of Medical Psychology, 32, 50-55.
Judd L L, Akiskal H S, Maser J D, Zeller P J, Endicott J, Coryell W, Paulus M P, Kunovac J L, Leon A C, Mueller T I, Rice J A, Keller M B. Major depressive disorder: a prospective study of residual subthreshold depressive symptoms as predictor of rapid relapse. J Affect Disord. 1998; 50(2-3):97-108
Judd L L, Paulus M J, Schettler P J, Akiskal H S, Endicott J, Leon A C, Maser J D, Mueller T, Solomon D A, Keller M B. Does incomplete recovery from first lifetime major depressive episode herald a chronic course of illness? Am J. Psychiatry. 2000; 157(9):1501-4.
Keller M B, Shapiro R W, Lavori P W, Wolfe N. Relapse in major depressive disorder: analysis with the life table. Arch Gen Psychiatry. 1982; 39(8):911-5
Labbate L A, Lare S B. Sexual dysfunction in male psychiatric outpatients: validity of the Massachusetts General Hospital Sexual Functioning Questionnaire. Psychother Psychosom. 2001; 70(4):221-5
Mcmillen, B.; Shank, J.; Jordan, K.; Williams, H.; Basile, A. (2007). “Effect of DOV 102,677 on the volitional consumption of ethanol by Myers' high ethanol-preferring rat”. Alcoholism, clinical and experimental research 31 (11): 1866-1871.
Miller I W, Keitner G I, Schatzberg A F, Klein D N, Thase M E, Rush A J, Markowitz J C, Schlager D S, Kornstein S G, Davis S M, Harrison W M, Keller M B. The treatment of chronic depression, part 3: psychosocial functioning before and after treatment with sertraline or imipramine. J Clin Psychiatry. 1998; 59(11):608-19
Montgomery S A, Åsberg M. A new depression scale designed to be sensitive to change. Br J Psychiatry 1979; 134:382-9
Montgomery, S. A. & Åsberg, M. (1979) A New Depression Scale Designed To Be Sensitive To Change. British Journal of Psychiatry. Vol. 1134, pp. 382-389.
Nitrogen Protecting Groups in Organic Synthesis, John Wiley and sons, New York, N.Y., 1981, Chapter 7; “Nitrogen Protecting Groups in Organic Chemistry”, Plenum Press, New York, N.Y., 1973, Chapter 2; See also, T. W. Green and P. G. M. Wuts in “Protective Groups in Organic Chemistry, 3rd edition” John Wiley & Sons, Inc. New York, N.Y., 1999.
Olsen, L. R., et al., The internal and external validity of the Major Depression Inventory in measuring severity of depressive states Psychological Medicine (2003), 33 : 351-356 Cambridge University Press.
Papakostas G I, Fava M, Thase M E. Treatment of SSRI-resistant depression: a meta-analysis comparing within-versus across-class switches. Biol Psychiatry. 2008 Apr. 1; 63(7):699-704
Paykel E S, Ramana R, Cooper Z, Hayhurst H, Kerr J, Barocka A. Residual symptoms after partial remission: an important outcome in depression. Psychol Med. 1995 November; 25(6):1171-80
Posner, K. (2008) Suicidality Across Drug Indications: Columbia Suicidal Adverse Event Identification and FDA Safety Concerns: The Issues and the Answers, From Outcomes to Feasibility. Invited presentation to the Center for Drug Evaluation and Research (CDER) at the Food and Drug Administration, Silver Spring, Md.
Posner K, Oquendo M, Gould M, et al. Columbia Classification Algorithm of Suicide Assessment (C CASA): classification of suicidal events in the FDA's pediatric suicidal risk analysis of antidepressants. Am J Psychiatry. 2007; 165:1035-1043.
Posner K, Oquendo M A, Gould M, Stanley B, Davies M (2007). Columbia Classification Algorithm of Suicide Assessment (C-CASA). Classification of Suicidal Events in the FDA's Pediatric Suicidal Risk Analysis of Antidepressants. Am J Psychiatry; 164:1035-1043
Povlock, S. L. and Amara, S. G., in “Neurotransmitter transporters: structure, function, and regulation,” Reith M E A, Editor, Humana Press, Totowa, pp. 1-28 (1997)
Quick Reference to the Diagnostic Criteria From DSM-IV (Diagnostic and Statistical Manual of Mental Disorders, Fourth Edition), The American Psychiatric Association, Washington, D.C., 1994 [0457] Perovic, S. and Muller, W. E., Arzneimittelforschung 45: 1145-1148 (1995)
Roth, B.; Sheffler, D.; Kroeze, W. (2004). “Magic shotguns versus magic bullets: selectively non-selective drugs for mood disorders and schizophrenia”. Nature reviews. Drug discovery 3 (4): 353-359.
Rush A J, Trivedi M H, Wisniewski S R, Nierenberg A A, Stewart J W, Warden D, Niederehe G, Thase M E, Lavori P W, Lebowitz B D, McGrath P J, Rosenbaum J F, Sackeim H A, Kupfer D J, Luther J, Fava M. Acute and longer-term outcomes in depressed outpatients requiring one or several treatment steps: a STAR*D report. Am J Psychiatry. 2006 November; 163(11):1905-17
Rush A J, Trivedi M H, Wisniewski S R, Stewart J W, Nierenberg A A, Thase M E, Ritz L, Biggs M M, Warden D, Luther J F, Shores-Wilson K, Niederehe G, Fava M; STAR*D Study Team. Bupropion-SR, sertraline, or venlafaxine-XR after failure of SSRIs for depression. N Engl J Med. 2006 Mar. 23; 354(12):1231-42.
Schrieber, R., R. Lew, L. Hardy, T. Cremers, K. Fang, U. Campbell; Poster 549.8/X2 Pharmacological characterization of the triple monoamine transporter uptake inhibitor SEP225289. Oct. 20, 2009.
Sepracor Press Release, Jul. 1, 2009 http://www.fiercebiotech.com/press-releases/sepracor-provides-update-clinical-trials-sep-225289-and-lunesta-r-pediatrics.
Sheehan D V, Y Lecrubier, K H Sheehan, P Amorim, J Janavs, E Weiller, T Hergueta, R Baker, G C Dunbar. The Mini-International Neuropsychiatric Interview (M.I.N.I.): the development and validation of a structured diagnostic psychiatric interview for DSM-IV and ICD-10. J Clin Psychiatry. 1998; 59 Suppl. 20:22-33; quiz 34-57.
Skolnick M, Popik P, Janowsky A, Beer B, Lippa A: Antidepressant-like actions of DOV 21,947: A triple reuptake inhibitor. European Journal of Pharmacology 2003 461:99-104
Skolnick, P. et al., Life Sci. 73: 3175-3179 (2003) [0447] Skolnick, P., et al., CNS Drug Reviews (2006)
Targum S D, Pollack M H, Fava M. Redefining affective disorders: relevance for drug development. CNS Neurosci Ther. 2008 Spring; 14(1):2-9
Thase M E, Entsuah A R, Rudolph R L. Remission rates during treatment with venlafaxine or selective serotonin reuptake inhibitors. Br J Psychiatry. 2001; 178:234-41
Thase M E, Simons A D, McGeary J, Cahalane J F, Hughes C, Harden T, Friedman E. Relapse after cognitive behavior therapy of depression: potential implications for longer courses of treatment. Am J Psychiatry. 1992; 149(8):1046-52
Thase M E. Evaluating antidepressant therapies: remission as the optimal outcome. J Clin Psychiatry. 2003; 64(Suppl 13):18-25
Tizzano, J.; Stribling, D.; Perez-Tilve, D.; Strack, A.; Frassetto, A.; Chen, R.; Fong, T.; Shearman, L. et al. (2008). “The triple uptake inhibitor (1R,5S)-(+)-1-(3,4-dichlorophenyl)-3-azabicyclo3.1.0 hexane hydrochloride (DOV 21947) reduces body weight and plasma triglycerides in rodent models of diet-induced obesity”. The Journal of pharmacology and experimental therapeutics 324 (3): 1111-1126.
Trivedi M H, Fava M, Wisniewski S R, Thase M E, Quitkin F, Warden D, Ritz L, Nierenberg A A, Lebowitz B D, Biggs M M, Luther J F, Shores-Wilson K, Rush A J; STAR*D Study Team. Medication augmentation after the failure of SSRIs for depression. N Engl J Med. 2006 Mar. 23; 354(12):1243-52
Trivedi M H, Rush A J, Ibrahim H M et al. The Inventory of Depressive Symptomatology, Clinician Rating (IDS-C) and Self-Report (IDS-SR), and the Quick Inventory of Depressive Symptomatology, Clinician Rating (QIDS-C) and Self-Report (QIDS-SR) in public sector patients with mood disorders: a psychometric evaluation. Psychol Med 2004; 34(1):73-82
U.S. Pat. No. 4,122,193; Scherm et al.; Oct. 24, 1978
U.S. Pat. No. 6,132,724; Blum; Oct. 17, 2000
U.S. patent application Ser. No. 11/371,178; Skolnick, et al.; filed Mar. 7, 2006
Van Londen L, Molenaar R P, Goekoop J G, Zwinderman A H, Rooijmans H G. Three- to 5-year prospective follow-up of outcome in major depression. Psychol Med. 1998; 28(3):731-5
Warren, W. L. Revised Hamilton Rating Scale for Depression (RHSD). (1994) Los Angeles, Western Psychological Services.
Zajecka J M. Treating depression to remission. J Clin Psychiatry. 2003; 64(Suppl 15):7-12

1. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof, wherein the (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof is substantially free of the corresponding (−) enantiomer.

2. The method of claim 1, wherein the pharmaceutical composition further comprises an additional psychotherapeutic agent, wherein the additional psychotherapeutic agent is an antidepressant, anti-psychotic, anti-convulsant or anxiolytic agent.

3. The method of claim 2, wherein the additional psychotherapeutic agent is a tri-cyclic antidepressant, specific monoamine reuptake inhibitor, selective serotonin reuptake inhibitor, selective norepinephrine or noradrenaline reuptake inhibitor, selective dopamine reuptake inhibitor, multiple monoamine reuptake inhibitor, monoamine oxidase inhibitor, atypical antidepressant, atypical antipsychotic, anticonvulsant, or opiate agonist.

4. The method of claim 1, wherein the (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof has no more than 2% w/w of the corresponding (−) enantiomer.

5. (canceled)

6. The method of claim 1, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane in crystalline form substantially free of other geometric, optical and polymorphic isomers thereof.

7-12. (canceled)

13. The method of claim 1, wherein the (+)-1-(3,4-dichlorophenyl)-3-azabicyclo[3.1.0]hexane agent is present in said oral unit dosage form in the amount of about 10 mg to about 300 mg.

14. (canceled)

15. The method of claim 1, wherein the effective amount is effective to decrease the human's score on a Montgomery Åsberg Depression Rating Scale to less than or equal to 12.

16-34. (canceled)

35. A method for treating depression in a human comprising administering to a human in need of treatment for depression a pharmaceutical composition comprising an effective amount of a (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof, wherein the (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof is substantially free of the corresponding (−) enantiomer and wherein the human in need of treatment for depression has previously been refractory to a prior course of treatment for depression.

36. The method of claim 35, wherein the pharmaceutical composition further comprises an additional psychotherapeutic agent, wherein the additional psychotherapeutic agent is an antidepressant, anti-psychotic, anti-convulsant or anxiolytic agent.

37. The method of claim 36, wherein the additional psychotherapeutic agent is a tri-cyclic antidepressant, specific monoamine reuptake inhibitor, selective serotonin reuptake inhibitor, selective norepinephrine or noradrenaline reuptake inhibitor, selective dopamine reuptake inhibitor, multiple monoamine reuptake inhibitor, monoamine oxidase inhibitor, atypical antidepressant, atypical antipsychotic, anticonvulsant, or opiate agonist.

38. The method of claim 35, wherein the (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof has no more than 2% w/w of the corresponding (−) enantiomer.

39. (canceled)

40. The method of claim 35, wherein the (+)-1-(3,4-dichlorophenyl)-3-azabicyclo[3.1.0]hexane agent is Polymorph A of an acid addition salt of (+)-1-(3,4-dichlorophenyl)-3-azabicyclo[3.1.0]hexane in crystalline form substantially free of other geometric, optical and polymorphic isomers thereof.

41. The method of claim 40, wherein the acid addition salt is a hydrochloride salt.

42. The method of claim 35, wherein the (+)-1-(3,4-dichlorophenyl)-3-azabicyclo[3.1.0]hexane agent is present in said oral unit dosage form in the amount of about 10 mg to about 300 mg.

43-46. (canceled)

47. The method of claim 35, wherein the human was refractory to treatment with an anti-depressant, wherein the anti-depressant is a tri-cyclic antidepressant, specific monoamine reuptake inhibitor, selective serotonin reuptake inhibitor, selective norepinephrine or noradrenaline reuptake inhibitor, selective dopamine reuptake inhibitor, norepinephrine-dopamine reuptake inhibitor, monoamine oxidase inhibitor, atypical antidepressant, atypical antipsychotic, anticonvulsants, or opiate agonist.

48. The method of claim 47, wherein the anti-depressant is a selective serotonin reuptake inhibitor.

49. The method of claim 35, wherein the individual failed to respond to a previous course of treatment.

50. The method of claim 35, wherein the individual did not achieve remission with a previous course of treatment.

51. The method of claim 35, wherein the individual had intolerable side effects from a previous course of treatment.

52. The method of claim 51, wherein the side effects are sexual dysfunction, weight gain, insomnia, dry mouth, constipation, nausea and vomiting, dizziness, memory loss, agitation, anxiety, sedation, headache, urinary retention, or abdominal pain

53. A method of treating depression comprising administering to a human in need of treatment an effective amount of a pharmaceutical agent comprising (+)-1-(3,4-dichlorophenyl)-3-azabicyclo[3.1.0]hexane agent comprising (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof, wherein the (+)-1-(3,4-dichlorophenyl)-3-azabicyclo[3.1.0]hexane or a pharmaceutically acceptable active salt, polymorph, glycosylated derivative, metabolite, solvate, hydrate, or prodrug thereof is substantially free of the corresponding (−) enantiomer, wherein administration of the pharmaceutical agent causes fewer side effects than administration of a composition comprising a balanced triple reuptake inhibitor.

54. The method of claim 53, wherein the side effect is a noradrenergic side effect.

55. The method of claim 54, wherein the noradrenergic side effect is substantially elevated heart rate or increased blood pressure.

56. The method of claim 53, wherein the side effect is a dopaminergic side effect.

57. The method of claim 56, wherein the side effect is nausea, vomiting, or hypomania.

58. The method of claim 53, wherein the (+)-1-(3,4-dichlorophenyl)-3-azabicyclo[3.1.0]hexane agent has improved anti-depressant effect in comparison to the balanced triple reuptake inhibitor.

59-92. (canceled)
